(19) United States
(12) Patent Application Publication
(10) Pub. No.: US 2024/0180956 A1
(43) Pub. Date: Jun. 6, 2024

(54) A VAGINAL CONTRACEPTIVE COMPOSITION FOR REINFORCEMENT OF THE CERVICAL MUCUS BARRIER PROPERTIES

(71) Applicant: CIRQLE BIOMEDICAL CONTRACEPTION APS, KØbenhavn N (DK)

(72) Inventors: Thomas CROUZIER, Solna (SE); Ulrike SCHIMPF, Stockholm (SE)

(21) Appl. No.: 18/283,757

(22) PCT Filed: Apr. 7, 2022
(86) PCT No.: PCT/DK2022/050071

§ 371 Date: Sep. 22, 2023

(30) Foreign Application Priority Data
Apr. 12, 2021 (DK) PA202170167

(51) Int. Cl. A61K 31/722; A61K 9/00; A61K 9/06; A61K 47/34; A61K 47/38; A61P 15/18

(52) U.S. Cl. CPC A61K 31/722; A61K 9/0034; A61K 9/06; A61K 47/34; A61K 47/38; A61P 15/18

(57) ABSTRACT

The disclosure relates to a vaginal contraceptive composition comprising one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group. The disclosure further relates to a use of a vaginal contraceptive composition, a vaginal contraceptive composition for use in therapy, a vaginal contraceptive composition for use as a contraceptive or contraceptive agent and a vaginal contraceptive composition for use in birth control or birth control therapy.

### TECHNICAL FIELD

The invention relates to a vaginal contraceptive composition comprising one or more active ingredients and at least one formulation compound selected from a physiological acceptable gelling agent or a physiological acceptable carrier, wherein at least one of the one or more active ingredients is a mucoadhesive polymer. The invention also relates to uses of such a vaginal contraceptive composition in therapy or contraception. The mucoadhesive polymer can cross-link the mucus layer without aggregating the mucus.

### BACKGROUND ART

The citation and incorporation of patent documents herein is done for convenience only and does not reflect any view of the validity, patentability, and/or enforceability of such patent documents.

There is a rapidly growing number of women that are unhappy with hormonal contraceptives but cannot find alternatives that are both convenient (no implantations, easy to use, flexible to use) and effective (over 90% efficacy in a typical use case). Indeed, 125 million couples in Europe and the US use birth control and hormonal contraceptives (pill, patch, implant, ring, etc.) are by far the most used birth control method. However, an increasing awareness of the side effects caused by hormones is upending the market for contraceptives. There are now robust evidence for side effects of hormonal contraceptives. Three studies including between 0.5 million and 1.8 million women show that the use of hormonal contraceptives increases women's rate of taking antidepressants by 23% and for teens the rate nearly doubles (Charlotte Wessel Skovlund, Lina Steinrud Mørch, Lars Vedel Kessing, and Øjvind Lidegaard. 2016. “Association of Hormonal Contraception With Depression.” JAMA Psychiatry 73 (11): 1154-62), increases women's rate of suicide attempt by 197% and the suicide rate by 308% (Charlotte Wessel Skovlund, Lina Steinrud Mørch, Lars Vedel Kessing, Theis Lange, and Øjvind Lidegaard. 2017. “Association of Hormonal Contraception With Suicide Attempts and Suicides.” The American Journal of Psychiatry, November) and increases the risk of developing breast cancer by 9% when used for less than a year and up to 38% when used for 10 years (Lina S. Mørch, Charlotte W. Skovlund, Philip C. Hannaford, Lisa Iversen, Shona Fielding, and Øjvind Lidegaard. 2017. “Contemporary Hormonal Contraception and the Risk of Breast Cancer.” The New England Journal of Medicine 377 (23): 2228-39). Many women now wish to move away from hormonal contraception, but they are unable to find suitable alternatives. The current alternatives are either inconvenient (condoms, diaphragms) or invasive (copper and hormone eluting implants) and can be poorly effective in actual use case (for instance condoms are only 85% effective on average).

There are over 400 square meters of epithelial surfaces hidden within the body of a human being, including the lung, gastrointestinal tract, and the female reproductive tract. The wet epithelial surfaces rely on a mucus gel for protection from dehydration, shear stress, and infections. Besides water, mucus mainly contains mucin biopolymers mixed with proteins, lipids, and salts. Mucins are large glycoproteins, which consist of an extended central protein core densely conjugated with oligosaccharides that can account for up to 50% of the molecule's molecular weight. Mucins have a central role in the protective function, creating a barrier, which serves as a size exclusion and affinity-based selective filter, preventing many deleterious molecules from reaching the epithelial surface.

Mucoadhesive polymers have been used for drug delivery due to their adhesive properties. For instance, they have been used to deliver drugs to inflammation sites.

Mucoadhesive polymers are typically assembled into materials or a gel alongside a drug, the intent being to concentrate the drug at the surface of the mucus layer and improve drug delivery.

WO2004069230 relates to pharmaceutical compositions containing a physiologically active agent, i.e. a drug, and a release sustaining or mucoadhesive agent e.g. chitosan, which serves to prolong the release of the active agent from the composition.

Another use for chitosan is in female contraception. One example of such may be found in CN102895256, which relates to chitosan gel foaming agent suitable for a female contraception and fungicidal effect and a preparation method thereof, and belongs to the technical field of foaming agent production. According to this disclosure, chitosan molecules are trapped in a solid foam matrix in association with polyacrylic acid, which physically prevents sperm passage. Additionally, the chitosan has a molecular weight distribution of 2,000-5,000 Da, a deacetylation degree above 95%, and a concentration of 5-10 wt. %, whereas the polyacrylic acid is in a concentration of 1-3 wt. %.

Another example of chitosan for female contraception may be found in WO2018185321, which relates to a mucoadhesive polymer, more specifically chitosan, which can cross-link the mucus layer without aggregating the mucus. The chitosan consisting of 4 to 20 monomer units and a deacetylation degree above 50%.

A third example may be found in U.S. Pat. No. 4,474,769, which relates to a method for injecting chitosan formulation directly in the uterine cavity of the female for a prolonged period to kill or inactivate mammalian spermatozoa.

It is known that mucoadhesive molecules promote the tightness and thickening of the mucosal tissue or enhance the barrier function, but usage has shown that the mucoadhesive polymers and the mucus-penetrating nanoparticles will cross-link and aggregate the mucus, leading to the formation of a highly swollen interpenetrating polymeric network. Aggregation of the mucus thus results in opening of pores within the mucus and causes a weakening of the barrier properties of the mucus. There is therefore still a need within the field for a composition, which shows improvement of cross-linking the mucus without aggregation.

Hence, it is an object of the invention to provide a mucoadhesive polymer, which can cross-link the mucus layer of the female cervical entrance, i.e. the exocervix, without aggregating the mucus, or with a lesser extent of aggregation compared to previous compositions in the art. The exocervix is the protective mucous membrane on the exterior of the cervix. Preferably, the mucoadhesive polymer may also cross-link the mucus layer of the female cervical entrance at the endocervix, which is the mucous membrane of the cervical canal. The cross-linking should be such that it is sufficient to prevent motile sperm from movement through the mucus layer.

Another object of the invention is to provide a mucoadhesive polymer, which provides a more reliable barrier effect for preventing cells and microorganisms such as bacteria, viruses, and/or spermatozoa to penetrate the cross-linked mucus and diffuse into the mucosa membrane.

Yet another object of the invention is to provide a mucoadhesive polymer, which provides a sufficient barrier effect for preventing pregnancy and/or sexually transmitted infections (STI).

### SUMMARY

In here is disclosed a newly developed technology that offers an alternative to hormonal contraception that is non-invasive convenient to use and effective. The strategy relies on making cervical mucus, the body's natural barrier between the vagina and the uterus, temporarily impenetrable for sperm cells. Cervical mucus protects women from infections and is mostly impenetrable to foreign cells throughout the month. However, around the day of ovulation, hormonal changes trigger a loosening of the mucus, which then becomes highly penetrable to sperm cells. It was found that the delivery of mucoadhesive (bio) polymers to the cervical mucus could alter the microstructure of the mucus gel and hereby reinforce the body's own natural barrier and prevent fertilization.

It has previously been stipulated that a low molecular weight mucoadhesive polymer (see e.g. WO2018185321 or Biomacromolecules, 2018, 19, 3, 872-882) containing between 4 and 20 monomers was an ideal mucoadhesive polymer to reinforce the mucus barrier properties by cross-linking said mucus. It was the opinion that the small size of the polymer advantageously allowed the molecule to diffuse inside the mucus. This should improve diffusion of the mucoadhesive polymer into the mucus membrane allowing it to cross-link the mucus layer over a large thickness, without aggregating the mucus. The small mucoadhesive polymers complex would thereby block the pores of the network and reinforce the barrier properties. However, such reinforcement of the barrier properties by e.g. chitosan was shown to work for small chitosan size in pig gastric mucins and colonic mucin cell lines, but for application as a contraceptive composition, the mucus that needs to be targeted is very different from the mucin in the gastrointestinal tracts. At ovulation, the cervical mucus is loose as to allow sperm passage through the gel. Compared to gastric or colonic mucus, the mucin content is decreased and the general mucus structure and composition is very different. It is therefore very important that the mucus layer is cross-linked enough to sufficiently prevent motile sperm from movement through the mucus layer but without aggregating the mucus.

Aggregation of the mucus is occurring when the mucin polymers condense around the mucoadhesive polymers. The result of this is the creation of areas of very dense mucin polymer aggregates and the creation of areas of very open and very loose mucin network. These open areas can allow sperm to go through.

Therefore, the present invention relates to a vaginal contraceptive composition comprising one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

Disclosed herein is shown that treatment with a 4 and 20 monomers mucoadhesive polymer as discussed above for gastric and colonic mucus is in fact not suitable for an effective reinforcement of the mucus barrier in the ovulatory mucus. Larger mucoadhesive polymers of at least 90,000 Da is shown to be much more effective at forming a barrier to e.g. sperm cells trying to penetrate the mucus barrier.

The composition as disclosed herein can efficiently be delivered to the cervix if e.g. formulated in a vaginal gel. The components of the gel could prevent diffusion of the mucoadhesive polymer from the gel into the mucus either by steric hindrance effects or by intermolecular interactions forming aggregates with the mucoadhesive polymer and gel, e.g. if the mucoadhesive polymer is provided as an excipient soft gel based on carboxymethyl cellulose (CMC), which is typically used as a gelling agent, this ingredient will strongly interact with e.g. chitosan, if used as the mucoadhesive polymer. At least two different types of gelling agents are suitable for vaginal formulation, natural and positively charged, without preventing penetration and/or cross-linking of the mucoadhesive polymer in the female ovulatory cervical mucus and without compromising the barrier reinforcement effect obtained by the mucoadhesive polymer. The gelling agents need to be either neutral or positively charged to avoid strong interactions with the mucoadhesive polymer. Another example is that is that if the mucoadhesive polymer interacts with the mucus via thiol groups, then the excipients should not contain thiol groups. Alternatively, the active ingredient could function in a way to also increase viscosity of the formulation, e.g. if using larger concentrations, and if this is the case, the formulation may not comprise a gelling agent but instead only utilize a physiological acceptable carrier to get all the active ingredients into solution and assist in delivering the correct concentrations. Further, the composition does not have to be in the form of a vaginal gel, but could e.g. be delivered at a vaginal film, a tablet, or a oil-based formulation.

The present invention further relates to the use of a vaginal contraceptive composition as a contraceptive agent, wherein said vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

The present invention also relates to a vaginal contraceptive composition for use in therapy, wherein the vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

Furthermore, the present invention relates to a vaginal contraceptive composition for use as a contraceptive or contraceptive agent, wherein the vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

The present invention relates even further to a vaginal contraceptive composition for use in birth control or birth control therapy, wherein the vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

Lastly, the present invention relates to a method of therapy, a method of avoiding pregnancy, a method of contraception, and/or a method of birth control or birth control therapy, wherein the method(s) comprises the steps of using a vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

### BRIEF DESCRIPTION OF THE DRAWINGS

FIG. 1—Sperm penetration through human cervical ovulatory mucus. The mucus was either non-treated (w/o), treated with 100 mM lactic acid solution (LAC) or treated with a chitosan designated as 95/50 and with a molecular weight of 102.3 kDa, dissolved in a 100 mM lactic acid solution.

FIG. 2—Sperm penetration through human cervical ovulatory mucus. The mucus was either non-treated (w/o), treated with 100 mM lactic acid solution (LAC) or treated with a chitosan designated as 95/100 and with a molecular weight of 150.0 kDa, dissolved in a 100 mM lactic acid solution.

FIG. 3—Sperm penetration through human cervical ovulatory mucus. The mucus was either non-treated (w/o), treated with 100 mM lactic acid solution (LAC) or treated with a chitosan designated as 95/100_2 and with a molecular weight of 175.6 kDa, dissolved in a 100 mM lactic acid solution.

FIG. 4—Sperm penetration through human cervical ovulatory mucus. The mucus was either non-treated (w/o), treated with 32.5 mM lactic acid solution (LAC) or treated with a chitosan designated as Z43 and with a molecular weight of 251.8 kDa, dissolved in a 32.5 mM lactic acid solution.

FIG. 5—Sperm penetration through human cervical ovulatory mucus. The mucus was either non-treated (w/o), treated with 100 mM lactic acid solution (LAC) or treated with a chitosan designated as 95/1000 and with a molecular weight of 290.9 kDa, dissolved in a 100 mM lactic acid solution.

FIG. 6—Sperm penetration through human cervical ovulatory mucus. The mucus was either non-treated (w/o), treated with 32.5 mM lactic acid solution (LAC) or treated with a chitosan designated as Z47 and with a molecular weight of 315.9 kDa, dissolved in a 32.5 mM lactic acid solution.

FIG. 7—Sperm counts in the distal cervix of ewes (A) or sperm counts in the uterus of ewes (B), both after administration of a formulation containing 95/50 chitosan (102.3 kDa), CsH (131.8 kDa), or no chitosan. The administration was followed by artificial insemination with fluorescently-labelled ram sperm, followed by the detection and counting of sperm by fluorescence endo-microscopy.

FIG. 8—Sperm penetration through human cervical ovulatory mucus. The mucus was either non-treated (w/o), treated with 32.5 mM lactic acid solution (LAC) or treated with a poly-L-lysine designated as PLL and with a molecular weight of 290.6 kDa, dissolved in a 32.5 mM lactic acid solution.

FIG. 9—Sperm penetration through cervical, ovulatory mucus: 0.5% (w/v) CO chitosan, in pH 5.5 solution containing only water (H₂O) (A), phosphate saline buffer (PBS) (B), or 100 mM lactic acid (LAC) (C).

FIG. 10—Sperm penetration through human cervical ovulatory mucus (A—7.1 kDa and B—18.9 kDa). (A) The mucus was either non-treated (w/o), treated with 100 mM lactic acid solution (LAC) or treated with a fungal based chitosan dissolved in a 100 mM lactic acid solution. (B) The mucus was either non-treated (w/o), treated with 32.5 mM lactic acid solution (LAC) or treated with a fungal based chitosan dissolved in a 32.5 mM lactic acid solution. The molar mass of the chitosan used are indicated on the left-hand side of the graphs.

### DETAILED DESCRIPTION

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context, e.g. a composition described herein as comprising a particular element should be understood as also describing a composition consisting of that element, unless otherwise stated or clearly contradicted by context. It will be further understood that the terms “comprises,” “comprising,” “includes” and/or “including,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms used herein (including technical and scientific terms) have the same meaning as commonly understood by those skilled in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined in the present specification.

As used herein, the singular forms “a,” “an,” and “the” are intended to include the plural forms, including “at least one,” unless the content clearly indicates otherwise. “At least one” is not to be construed as limiting “a” or “an.”

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

When describing the below embodiments, aspects, and definitions, the present invention envisages all disclosed embodiments and definitions in combination with all the disclosed aspects. Additionally, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

Disclosed in a first aspect of the present invention is a vaginal contraceptive composition comprising one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

Disclosed in a second aspect of the present invention is the use of a vaginal contraceptive composition as a contraceptive agent, wherein said vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

Disclosed in a third, fourth, and fifth aspect of the present invention is a vaginal contraceptive composition for use in therapy, for use as a contraceptive or contraceptive agent, and for use in birth control or birth control therapy, wherein the vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

Disclosed in a sixth, seventh, eighth, and ninth aspect of the present invention is a method of therapy, a method of avoiding pregnancy, a method of contraception, and a method of birth control or birth control therapy, wherein the method(s) comprises the steps of using a vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

As disclosed herein, a contraceptive composition is a composition, which prevents a female from getting pregnant by preventing the spermatozoa from reaching the ovum or ova, hereby keeping the egg cell and sperm cell apart via a barrier method that may additionally help to protect against sexually transmitted infections. The spermatozoa is prevented from reaching the ovum or ova by creating a barrier at the exocervix and endocervix, hereby keeping the sperm cells in the vagina (or vaginal tract). The sperm cells will therefore never enter the uterus through the cervix and hereby have the opportunity to enter the fallopian tube (or uterine tube) to reach the egg. They are instead left to be killed by the acidic fluids inside the vagina or lost in “flow-back”.

As disclosed herein, an active ingredient is one or more compounds in the contraceptive composition, which is providing the contraceptive ability, i.e. preventing a female from getting pregnant by preventing the spermatozoa from reaching the ovum or ova.

A mucoadhesive polymer is a polymer, which shows mucoadhesion. Mucoadhesion is here described as the interfacial forces that hold together two biological materials, such as the attractive forces between a biological material and mucus or a mucus membrane. By mucoadhesive polymer, is therefore meant a polymer, which has attractive force towards mucus or a mucus membrane.

Mucus is the protective cover of all epithelial surfaces, which keep the epithelial layer moist and prevent microorganism from invading the epithelium. A natural protective effect is achieved because the mucus traps microorganisms and facilitates their distal transport. When mentioning the barrier effect achieved by the mucoadhesive polymer, it is the reinforcement of the mucus due to cross-linking of the polymer. The effect of the reinforced barrier is based on the tightness of the cross-linked mucus, which stops diffusion, and how long the mucus is reinforced by the complexed mucoadhesive polymer. The latter is determined by the natural turnover of the mucus secreted by cells from the mucosa, which removes the mucus comprising the cross-linked polymer.

The mucus layer on the mucosa of the cervix differs in rheological properties depending on the four phases of the menstrual cycle. At ovulation, the cervical mucus is loose as to allow sperm passage through the gel, and therefore the pore size of the mucus will also be increased. The thickness of the barrier layer may be tailored to be impermeable to relatively large cells, such as spermatozoa, however it may also be tailored to an even tighter barrier layer, which may be required to be impermeable to bacteria, viruses, or other microorganisms or pathogens.

Given the effective barrier that the thickening of cervical mucus can form, it is well accepted that cervical mucus barrier properties can be leveraged as a contraceptive method. Indeed, the primary mechanism for contraception of the levonorgestrel intrauterine system (LNG-IUS) and progestin-only mini-pill are through to be cervical mucus thickening. The approach as disclosed in here would differ from these approaches not by the nature of the barrier, but by the mean to create the barrier: a non-hormonal, non-invasive, on demand contraceptive free from side effects.

The mucoadhesive polymer therefore provides a more reliable barrier effect, which prevents cells and microorganisms such as bacteria, viruses, and spermatozoa from penetrating the cross-linked mucus and diffuse into the mucosa membrane. The vaginal contraceptive composition may make such tight cross-linked mucus that it prevents even the smallest microorganisms from penetration, hereby not only preventing pregnancy but also sexually transmitted infections (STI).

Hence, in one or more embodiments according to any aspect, the composition is a contraceptive composition. Moreover, the invention provides a contraceptive composition free from hormones or chemicals that have undesired side effects. Undesired effects may include emboli, migraine, or minor side effects such as influencing the menstrual cycle. The effective time of the mucoadhesive polymer according to the invention is determined by the turnover of mucus, which means that the contraceptive effect is temporary. It may also be determined by the thickness of the barrier created. The contraceptive dissolves after the effective time and the fertility is unaffected. The time of sufficient contraception is affected by several factors such as the biological turnover of the mucus, the concentration of mucoadhesive polymer, the size of the mucoadhesive polymer, etc. The contraceptive effect last for a period of time, such as 1, 2, 3, 4, 5, 6, 7, 8, 9, 10 hours, 1, 2, 3 day(s) or even up to 10 days, which is a sufficient time to block sperm cells from entering the cervix. By blocking the sperm from entering the cervix, the acidic environment of the vagina will reduce the motility of the sperm and weaken the sperm leaving them unable to fertilise an egg. Under natural conditions, the sperm cells will need to enter the cervix within minutes to survive. The full contraceptive effect is gained from a single application, which means that non-coherent use of the contraceptive composition gives the same protection as coherent use. That the full contraceptive effect is gained from a single application may also mean that temporary contact of the cervical mucus with the contraceptive composition may give the same full protection as the continued contact with the cervical mucus.

The size of the mucoadhesive polymer may allow the molecule to diffuse inside the mucus. This diffusion of the mucoadhesive polymer into the mucus membrane allows that it can cross-link the mucus layer over a large thickness sufficiently to prevent motile sperm from movement through the mucus, without aggregating the mucus. Alternatively, the size of the mucoadhesive polymer is so large that it only diffuses slightly into the pore of the mucus, but instead creates a cross-linked barrier at the outside of the mucus layer. Either way, the mucoadhesive polymers complex to the mucus thereby blocking the pores of the network and reinforcing its barrier properties. At ovulation, the cervical mucus is loose as to allow sperm passage through the gel, and therefore the pore size of the mucus will also be increased as comparted with non-ovulatory mucus. The size of the mucoadhesive polymer should therefore be tailored towards this increased pore size of ovulatory cervical mucus. A size of the mucoadhesive polymer working for the increased pore size of ovulatory cervical mucus is also effectively working when the pore size of the cervical mucus is not increased due to ovulation.

Moreover, the size of the mucoadhesive polymers are generally more soluble at lower molecular weight, and has less steric hindrance. If the mucoadhesive polymer is large it may not pass, or fit, through the pores of the mucus and it will therefore end up interacting with and increased number of mucin molecules hereby not diffusing through the gel, but if it is too small, it may pass straight through without interacting with any mucin molecules. Hence, a compromise between a larger size, but not too large, to avoid the mucoadhesive polymer to penetrate all the way through the pore of the mucus, and a smaller size to obtain a high enough solubility for appropriate delivery to a mucus membrane of a subject is needed.

Hereby, the mucoadhesive polymer can be delivered more efficiently to the mucus membrane, which in turn allows a stronger and effective cross-linking.

The mucoadhesive polymer is generally cationic with at least 50% of the monomers being charged. The monomer units may e.g. comprise amino groups, which at physical pH is positively charged. It may also be hydrophobic, e.g. with up to 50% of the monomers having a hydrophobic side chains. These two features of the mucoadhesive polymer may play a roll, if selecting a compatible excipient.

Amino groups, in chemistry, are functional groups that consists of a nitrogen atom attached by single bonds to hydrogen atoms, alkyl groups, aryl groups, or a combination of these three. An organic compound that contains an amino group is called an amine. Amines are derivatives of the inorganic compound ammonia, NH₃. When one, two, or all three of the hydrogens in ammonia are replaced by an alkyl or aryl group, the resulting compound is known as a primary, secondary, or tertiary amine, respectively. Like ammonia, the amines are weak bases because the unshared electron pair of the nitrogen atom can form a coordinate bond with a proton. A water-insoluble amine can be made dissolvable by adding acid to form its water-soluble amine salt. The amino groups make the mucoadhesive polymer basic, which is advantageous for their binding to the mucus membrane because of the large amount of negatively charged molecules is contains. The basic amino groups particularly provide a more efficient cross-linking. In addition, when some of the monomers of the mucoadhesive polymer, up to 50%, comprise a hydrophobic group, the mucoadhesive polymer can also adhere to and diffuse into the mucus membrane to cross-link the mucus membrane without aggregating the mucus. In the context of the invention, an amino group is —NH₂, where one or both hydrogen atoms may be substituted with a group R, or the amino group may be a quaternary amino group with three R groups, i.e. —N⁺R₃. R may be selected from C1-C4 alkyls, optionally substituted with one or more —OH, —SH, or —NH₂. When more than one R is present on the same nitrogen atom these may be the same or different R groups. As long as R has four or fewer carbon atoms, and in particular when hydrogen atoms of the R groups are substituted by one or more —OH, —SH, or —NH₂, the amino groups as disclosed herein are generally seen as being basic. Longer alkyl chains, e.g. having five or more carbon atoms, may mask the basicity of the amino group, however, amino groups carrying alkyls of five or more carbon atoms are still counted as amino groups in the context of the present invention. Likewise, the sugar may also comprise an amide group, e.g. —CONHCH₃, or —NHCHO, but such groups are not counted as amino groups in the context of the invention.

In one or more embodiments according to any aspect, the amino groups do not comprise alkyls of ten or more carbon atoms.

In one or more embodiments according to any aspect, the amino groups do not comprise alkyls of nine or more carbon atoms.

In one or more embodiments according to any aspect, the amino groups do not comprise alkyls of eight or more carbon atoms.

In one or more embodiments according to any aspect, the amino groups do not comprise alkyls of six or more carbon atoms.

In one or more embodiments according to any aspect, the amino groups do not comprise alkyls of five or more carbon atoms.

In one or more embodiments according to any aspect, at least 55% of the monomer units, such as at least 60% of the monomer units, such as at least 65% of the monomer units, such as at least 70% of the monomer units comprise at least one amino group.

In one or more embodiments according to any aspect, one or more of the at least one amino group is a primary amine. In the context of the invention, a primary amine is an amino group without any hydrogen atoms being substituted with a group R substitution (i.e. —NH₂).

In one or more embodiments according to any aspect, the at least one amino group is a primary amine.

In one or more embodiments according to any aspect, the vaginal contraceptive composition has an osmolality of at least 50 mOsm/kg, such as at least 75 mOsm/kg, or such as at least 100 mOsm/kg. In one or more embodiments according to any aspect, the vaginal contraceptive composition has an osmolality of at least 0.050 Osm/kg, such as at least 0.075 Osm/kg, or such as at least 0.100 Osm/kg.

Osmolarity and osmolality are frequently confused and incorrectly interchanged. Osmolarity refers to the number of solute particles per 1 L of solvent, whereas osmolality is the number of solute particles in 1 kg of solvent. For dilute solutions, the difference between osmolarity and osmolality is insignificant. Measurements of osmolarity are temperature dependent because the volume of solvent varies with temperature (i.e., the volume is larger at higher temperatures). In contrast, osmolality, which is based on the mass of the solvent, is temperature independent. For this reason, osmolality is the preferred term for biologic systems. Osmolality has the units of Osm/kg solvent (e.g. H₂O). Because of the dilute nature of physiologic solutions and because water is usually the solvent, osmolalities may also be expressed as milliosmoles per kilogram of solvent (mOsm/kg). These values allow the measurement of the osmotic pressure of a solution and the determination of how the solvent will diffuse across a semipermeable membrane (osmosis) separating two solutions of different osmotic concentration. Ionic compounds, such as salts, can dissociate in solution into their constituent ions, so there is not a one-to-one relationship between the molality and the osmolality of a solution. For example, sodium chloride (NaCl) dissociates into Na⁺ and Cl⁻ ions. Thus, for every 1 mole of NaCl in solution, there are 2 osmoles of solute particles (i.e., a 1 mol/L NaCl solution is a 2 osmol/L NaCl solution). Both sodium and chloride ions affect the osmotic pressure of the solution. Another example is magnesium chloride (MgCl₂), which dissociates into Mg²⁺ and 2Cl⁻ ions. For every 1 mole of MgCl₂ in the solution, there are 3 osmoles of solute particles. Nonionic compounds do not dissociate, and form only 1 osmole of solute per 1 mole of solute. For example, a 1 mol/L solution of glucose is 1 osmol/L. Multiple compounds may contribute to the osmolarity/osmolality of a solution. For example, a 3 Osm solution might consist of: 3 moles glucose, or 1.5 moles NaCl, or 1 mole glucose+1 mole NaCl, or 2 moles glucose+0.5 mole NaCl, or any other such combination.

To obtain a given osmolality salts like NaCl or buffer components can be added to the composition. Other excipients like the gelling agents, carriers, glycerol, polyethylene glycol, and sugars, like glucose or fructose, may also be added to the composition to obtain a targeted osmolality.

There are clinical and non-clinical evidences that variation away from the osmolality of vaginal fluids leads to an irritation of the vaginal epithelium. The typical vaginal fluid osmolality varying during the menstrual cycle between 300-480 mOsm/kg. Hyperosmolar vaginal formulation in particular were shown to be cytotoxic and lead to increase in the transmission risk of genital herpes infections in the mouse model. Hence, hyper-osmolality needs to be avoided, such as ranges above 3000 mOsmol/kg.

The osmolarity of the present composition can be measured using an osmometer, which measures colligative properties, such as freezing-point depression, vapour-pressure lowering, or boiling-point elevation. Alternatively, the osmolarity can be calculated based on the compounds in the composition.

In one or more embodiments according to any aspect, the vaginal contraceptive composition has an osmolality of less than 3.000 Osmol/kg, such as less than 2.000 Osmol/kg, such as less than 1.000 Osmol/kg, such as less than 0.750 Osmol/kg, or such as less than 0.500 Osmol/kg.

In one or more embodiments according to any aspect, the vaginal contraceptive composition has an osmolality between 0.050 Osmol/kg and 3.000 Osmol/kg, such as between 0.050 Osmol/kg and 2.000 Osmol/kg, such as between 0.050 Osmol/kg and 1.000 Osmol/kg, such as between 0.050 Osmol/kg and 0.750 Osmol/kg, or such as between 0.050 Osmol/kg and 0.100 Osmol/kg. All end-points are included in the above ranges.

In one or more embodiments according to any aspect, the vaginal contraceptive composition has an osmolality between 0.050 Osmol/kg and 3.000 Osmol/kg, such as between 0.100 Osmol/kg and 3.000 Osmol/kg, such as between 0.750 Osmol/kg and 3.000 Osmol/kg, such as between 1.000 Osmol/kg and 3.000 Osmol/kg, or such as between 2.000 Osmol/kg and 3.000 Osmol/kg. All end-points are included in the above ranges.

In one or more embodiments according to any aspect, the osmolarity is obtained by the composition comprising saline water in a concentration of at least 5 mM, such as least 10 mM, such as least 15 mM, such as least 20 mM, such as least 25 mM, such as least 50 mM, such as least 75 mM, or such as least 100 mM.

Saline water (or salt water) is water that contains a high concentration of dissolved salts, such as but not limited to sodium chloride. The disclosed saline water concentration corresponds to the resulting concentration of salt in the final formulation together with the active compound.

In one or more embodiments according to any aspect, the osmolarity is obtained by the composition comprising saline water in a concentration of less than 500 mM, such as less than 450 mM, such as less than 400 mM, such as less than 350 mM, such as less than 300 mM, such as less than 250 mM, such as less than 200 mM, or such as less than 175 mM.

In one or more embodiments according to any aspect, the osmolarity is obtained by the composition comprising saline water in a concentration of between 5 and 500 mM, such as between 10 and 450 mM, such as between 15 and 400 mM, such as between 20 and 350 mM, such as between 25 and 300 mM, such as between 50 and 250 mM, such as between 75 and 200 mM, or such as between 100 and 175 mM.

The vaginal contraceptive composition comprises one or more active ingredients and at least one formulation compound selected from a physiological acceptable gelling agent or a physiological acceptable carrier. The one or more active ingredients may be administered in a physiologically acceptable gelling agent or carrier, which ensures that the one or more active ingredients are soluble in the conditions where it is used and ensures that the one or more active ingredients are evenly distributed in the target area. Here evenly distributed means that the targeted mucus area is subjected to at least a minimum amount of composition with enough active ingredient to diffuse into the mucus and reinforce the mucus barrier.

Thus, in one or more embodiments according to any aspect, the one or more active ingredients is administered in at least one physiologically acceptable gelling agent. In one or more embodiments according to any aspect, the composition comprising one or more active ingredients and a physiological acceptable gelling agent. In one or more embodiments, according to any aspect, the at least one formulation compound is a physiological acceptable gelling agent.

By physiological acceptable gelling agent is meant a non-toxic compound, which in an effective dose, is neither chemically nor physically toxic to a human and/or animal organism.

In one or more embodiments according to any aspect, the physiological acceptable gelling agent is selected from hydroxyethyl cellulose (HEC), glycerol, polyols such as mannitol or sorbitol, hydroxypropylmethyl cellulose (HPMC), hydroxypropyl cellulose, guar gum, or combinations hereof. Any suitable pharmaceutical gelling agent may be used as long as the gelling agent does not interact with the one or more active ingredients, especially the mucoadhesive polymer.

By combination with a physiological acceptable gelling agent, the contact area between the composition and the mucus is maximised. Increased contact area may help ensure that a maximum amount of mucoadhesive polymer can diffuse into the mucus layer and modify its properties. Also contributing to the increased diffusion is a high density of the composition. By having a high composition density, e.g. similar to that of water, such as in a semi-solid gel, the applied composition is able to change shape and envelope the full surface of the cervical entrance.

Hydroxyethyl cellulose (or ethyl cellulose) is a gelling and thickening agent derived from cellulose. It is widely used in cosmetics, cleaning solutions, and other household products. Hydroxyethyl cellulose and hydroxymethyl cellulose (or methyl cellulose) are frequently used with hydrophobic drugs in capsule formulations, to improve the drugs' dissolution in the gastrointestinal fluids. This process is known as hydrophilization.

In one or more embodiments according to any aspect, the physiological acceptable gelling agent is selected from hydroxyethyl cellulose, hydroxymethyl cellulose, or combinations hereof.

Glycerol, also called glycerine or glycerin, is a simple polyol compound. It is a colourless, odourless, viscous liquid, which is sweet tasting and non-toxic. The glycerol backbone is found in many lipids, which are known as glycerides. It is widely used in the food industry as a sweetener and humectant in pharmaceutical formulations. Glycerol has three hydroxyl groups that are responsible for its solubility in water and its hygroscopic nature.

In one or more embodiments according to any aspect, the physiological acceptable gelling agent is glycerol.

Hydroxypropyl methylcellulose (HPMC), also called hypromellose, is a semisynthetic, inert, viscoelastic polymer used as eye drops, as well as an excipient and controlled-delivery component in oral medicaments, found in a variety of commercial products. As a food additive, hypromellose is an emulsifier, thickening, and suspending agent, and an alternative to animal gelatine. Its Codex Alimentarius code (E number) is E464.

In one or more embodiments according to any aspect, the physiological acceptable gelling agent is hydroxypropylmethyl cellulose (HPMC).

Hydroxypropyl cellulose (HPC) is a derivative of cellulose with both water solubility and organic solubility. It is used as an excipient, and topical ophthalmic protectant and lubricant. HPC is an ether of cellulose in which some of the hydroxyl groups in the repeating glucose units have been hydroxypropylated forming —OCH₂CH(OH)CH₃ groups using propylene oxide. The average number of substituted hydroxyl groups per glucose unit is referred to as the degree of substitution (DS). Complete substitution would provide a DS of 3. Because the hydroxypropyl group added contains a hydroxyl group, this can also be etherified during preparation of HPC. When this occurs, the number of moles of hydroxypropyl groups per glucose ring, moles of substitution (MS), can be higher than 3.

In one or more embodiments according to any aspect, the physiological acceptable gelling agent is hydroxypropyl cellulose.

Guar gum, also called guaran, is a galactomannan polysaccharide extracted from guar beans that has thickening and stabilizing properties useful in the food, feed, and industrial applications. The guar seeds are mechanically dehusked, hydrated, milled, and screened according to application. It is typically produced as a free-flowing, off-white powder. Chemically, guar gum is an exo-polysaccharide composed of the sugars galactose and mannose. The backbone is a linear chain of β 1, 4-linked mannose residues to which galactose residues are 1, 6-linked at every second mannose, forming short side-branches. Guar gum has the ability to withstand temperatures of 80° C. for five minutes.

In one or more embodiments according to any aspect, the physiological acceptable gelling agent is guar gum.

In one or more embodiments according to any aspect, the physiological acceptable gelling agent is in a concentration of between 0.05 wt. % and 50.0 wt. % of the total weight of the vaginal contraceptive composition, such as between 0.10 wt. % and 50.0 wt. %, such as between 0.10 wt. % and 40.0 wt. %, such as between 0.10 wt. % and 30.0 wt. %. such as between 0.10 wt. % and 20.0 wt. %, such as between 0.05 wt. % and 10.0 wt. %, such as between 0.10 wt. % and 10.0 wt. %, or such as between 0.50 wt. % and 10.0 wt. % of the total weight of the vaginal contraceptive composition.

In one or more embodiments according to any aspect, the one or more active ingredients is administered in at least one physiologically acceptable carrier. In one or more embodiments according to any aspect, the composition comprising one or more active ingredients and a physiologically acceptable carrier. In one or more embodiments according to any aspect, the at least one formulation compound is a physiological acceptable carrier.

By physiological acceptable carrier is meant a non-toxic compound, which in an effective dose, is neither chemically nor physically toxic to a human and/or animal organism.

In one or more embodiment according to any aspect, the pharmaceutical acceptable carrier is selected from water, dimethyl sulfoxide (DMSO), saline (saline solution), or a combination thereof.

In one or more embodiments according to any aspect, the physiological acceptable carrier is in a concentration of between 5.0 wt. % and 99.0 wt. % of the total weight of the vaginal contraceptive composition, such as between 5.0 wt. % and 90.0 wt. %, such as between 5.0 wt. % and 80.0 wt. %, such as between 10.0 wt. % and 75.0 wt. %, such as between 10.0 wt. % and 70.0 wt. %. such as between 10.0 wt. % and 60.0 wt. %, such as between 10.0 wt. % and 50.0 wt. %, such as at least 5.0 wt. %, or such as at least 10.0 wt. % of the total weight of the vaginal contraceptive composition.

In one or more embodiments according to any aspect, the one or more active ingredients is administered in at least one physiologically acceptable gelling agent, and at least one physiologically acceptable carrier. In one or more embodiments according to any aspect, the composition comprising one or more active ingredients, a physiological acceptable gelling agent, and a physiologically acceptable carrier. In one or more embodiments according to any aspect, the at least one formulation compound is at least a physiological acceptable gelling agent and a physiological acceptable carrier.

In one or more embodiments according to any aspect, the vaginal contraceptive composition is not a foam.

A foam is an object formed by trapping pockets of gas in a liquid or solid. In most foams, the volume of gas is large, with thin films of liquid or solid separating the regions of gas.

The mucoadhesive polymer may be a polysaccharide where C6 sugars are linked to each other via ether, ester, or amide bonds. The monomers of C6 sugars may be linked via e.g. any ether bond, e.g. C₁ and C₄ of two adjacent C6 sugars may be linked, or C₁ and C₆ of two adjacent C6 sugars may be linked. In particular, when the monomer is a C6 sugar, e.g. glucose, the monomers, e.g. glucose monomers, may be linked via β 1, 4-linkages.

The at least one amino group may be linked to any carbon atom of the glucose monomers, e.g. C₂ or C₃.

In one or more embodiments according to any aspect, the mucoadhesive polymer consists of a plurality of monomer units linked to each other via ether bonds.

In one or more embodiments according to any aspect, the monomer units are selected from C6 sugars, amino-functionalised C6 sugars, or combinations hereof.

C6 sugars are carbohydrates whose molecules have six carbons (i.e. hexoses). The best-known example of this class is glucose, a principal component of cellulose and starch molecules.

An amino-functionalized C6 sugar (or amino sugar) is a sugar molecule in which a hydroxyl group has been replaced with an amine group. More than 60 amino sugars are known, with one of the most abundant being N-acetyl-D-glucosamine, which is the main component of chitin. The amino-functionalization may be on the C₂, C₃, C₄, and/or C₆ of the C6 sugar.

In one or more embodiments according to any aspect, the monomer units are amino-functionalised C6 sugars.

In one or more embodiments according to any aspect, the monomer units are a combination of D-glucosamine and N-acetyl-D-glucosamine.

D-glucosamine (C₆H₁₃NO₅) is an amino sugar and a prominent precursor in the biochemical synthesis of glycosylated proteins and lipids. D-Glucosamine is part of the structure of the polysaccharides, chitosan, and chitin. D-Glucosamine is one of the most abundant monosaccharides. It is produced commercially by the hydrolysis of crustacean exoskeletons or, less commonly, by fermentation of a grain such as corn or wheat.

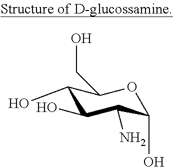

N-Acetyl-D-glucosamine (GlcNAc, C₈H₁₅NO₆) is a monosaccharide and a derivative of glucose. It is significant in several biological systems. It is part of a biopolymer in the bacterial cell wall, which is built from alternating units of GlcNAc and N-acetylmuramic acid (MurNAc), cross-linked with oligopeptides at the lactic acid residue of MurNAc. This layered structure is called peptidoglycan (formerly called murein). GlcNAc is the monomeric unit of the polymer chitin, which forms the outer coverings of insects and crustaceans.

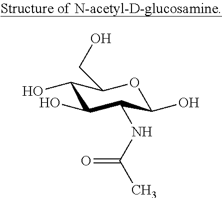

In one or more embodiments according to any aspect, at least 50% of the monomer units is D-glucosamine whereas 50% or less of the monomer units is N-acetyl-D-glucosamine, such as between 50% and 100% is D-glucosamine and between 0% and 50% is N-acetyl-D-glucosamine.

By at least 50% of the monomer units is D-glucosamine is meant that at least 50% of the total amount of monomers in the mucoadhesive polymer is from D-glucosamine. Similarly, by 50% or less of the monomer units is N-acetyl-D-glucosamine is meant that 50% or less of the total amount of monomers in the mucoadhesive polymer is from N-acetyl-D-glucosamine. Further, by between 50% and 100% or between 0% and 50% is meant that between 50% and 100% or between 0% and 50% of the total amount of monomers in the mucoadhesive polymer is from said monomer unit, all end-points are included (0%, 50%, and 100%).

In one or more embodiments according to any aspect, at least 65% of the monomer units is D-glucosamine whereas 35% or less of the monomer units is N-acetyl-D-glucosamine, such as between 65% and 100% is D-glucosamine and between 0% and 35% is N-acetyl-D-glucosamine. End-points are included.

In one or more embodiments according to any aspect, the mucoadhesive polymer is chitosan where at least 50% of the glucose monomers have an —NH₂ group. The chitosan may also be referred to as at least 50% deacetylated.

Chitosan is a linear polysaccharide composed of randomly distributed β 1, 4-linked D-glucosamine (deacetylated unit) and N-acetyl-D-glucosamine (acetylated unit). It is made by treating the chitin shells of shrimp and other crustaceans with an alkaline substance, like sodium hydroxide, or it can be extracted from other sources such as fungal cell walls.

When the mucoadhesive polymer is chitosan it is important to avoid e.g. the presence of high molecular weight polyacrylic acids, as the carboxyl functional groups in the acrylic monomers form ionic complexes with the basic amino groups in the chitosan chain, which leads to the formation of a highly swollen interpenetrating polymeric network. This will result in aggregation of the mucus thus results in opening of pores within the mucus and causes a weakening of the barrier properties of the mucus.

Chitosan is a strongly mucoadhesive molecule, meaning that it is able to entangle with and bind to the mucin glycoproteins that compose the mucus gels. As such, they have been used in mucosal drug delivery devices and are included in commercial hemostatic products.

However, chitosan used as disclosed herein is typically of high molar mass, and thus diffuse poorly within the mucus gel and tend to aggregate and compact the mucus.

In one or more embodiments according to any aspect, the mucoadhesive polymer is chitosan where at least 50% of the glucose monomers have an —NH₂ group, and where 40% or less of the glucose monomers have a —CONHCH₃ group.

In one or more embodiments according to any aspect, the mucoadhesive polymer is chitosan where at least 50% of the glucose monomers have an —NH₂ group, and where 20% or less of the glucose monomers have a —CONHCH₃ group.

In one or more embodiments according to any aspect, the mucoadhesive polymer is chitosan where at least 70% of the glucose monomers have an —NH₂ group. The chitosan may also be referred to as at least 70% deacetylated.

In one or more embodiments according to any aspect, the mucoadhesive polymer is chitosan where at least 70% of the glucose monomers have an —NH₂ group, and where 20% or less of the glucose monomers have a —CONHCH₃ group.

In one or more embodiments according to any aspect, the mucoadhesive polymer is selected from chitosan, chitosan-trimethyl, chitosan-thioglycolic acid, chitosan-iminothiolane, chitosan-thioethylamidine, or combinations hereof.

Chitosan-trimethyl is a quaternized hydrophilic derivative of chitosan. This quaternized derivative of chitosan possesses a positive charge and is soluble over a wide range of pH.

Chitosan-thioglycolic acid, chitosan-iminothiolane, and chitosan-thioethylamidine are chitosan-derivatives, which have been modified by the introduction of different thiol groups. The thiol groups are introduced to chitosan via amide bond formation mediated by a carbodiimide. The properties of the resulting polymer is hereby altered in regard to water solubility, mucoadhesion, biodegradability and in situ gelling compared to the original polymer.

In one or more embodiments according to any aspect, the mucoadhesive polymer is chitosan which has a molecular weight between 90,000 Da and 350,000 Da. In one or more embodiments according to any aspect, the mucoadhesive polymer has a molecular weight between 100,000 Da and 350,000 Da, such as between 100,000 Da and 300,000 Da, such as between 100,000 Da and 275,000 Da, such as between 100,000 Da and 250,000 Da, such as between 101,000 Da and 250,000 Da, or such as between 105,000 Da and 250,000 Da. All end-points are included in the above ranges.

In one or more embodiments according to any aspect, the mucoadhesive polymer has a molecular weight between, such as between 90,000 Da and 340,000 Da, such as between 90,000 Da and 330,000 Da, such as between 90,000 Da and 325,000 Da, such as between 90,000 Da and 320,000 Da, such as a molecular weight between 90,000 Da and 310,000 Da, such as a molecular weight between 90,000 Da and 300,000 Da, such as a molecular weight between 90,000 Da and 275,000 Da, or such as a molecular weight between 90,000 Da and 250,000 Da. All end-points are included in the above ranges.

In one or more embodiments according to any aspect, the mucoadhesive polymer has a molecular weight between, such as between 100,000 Da and 340,000 Da, such as between 100,000 Da and 330,000 Da, such as between 100,000 Da and 325,000 Da, such as between 100,000 Da and 320,000 Da, such as a molecular weight between 100,000 Da and 310,000 Da, such as a molecular weight between 100,000 Da and 300,000 Da, such as a molecular weight between 100,000 Da and 275,000 Da, such as a molecular weight between 100,000 Da and 250,000 Da, or such as a molecular weight between 100,000 Da and 200,000 Da. All end-points are included in the above ranges.

In one or more embodiments according to any aspect, the mucoadhesive polymer has a molecular weight between, such as between 101,000 Da and 340,000 Da, such as between 101,000 Da and 330,000 Da, such as between 101,000 Da and 325,000 Da, such as between 101,000 Da and 320,000 Da, such as a molecular weight between 101,000 Da and 310,000 Da, such as a molecular weight between 101,000 Da and 300,000 Da, such as a molecular weight between 101,000 Da and 275,000 Da, such as a molecular weight between 101,000 Da and 250,000 Da, or such as a molecular weight between 101,000 Da and 200,000 Da. All end-points are included in the above ranges.

In one or more embodiments according to any aspect, the mucoadhesive polymer has a molecular weight between 102,000 Da and 350,000 Da, such as between 102,000 Da and 340,000 Da, such as between 102,000 Da and 330,000 Da, such as between 102,000 Da and 325,000 Da, such as between 102,000 Da and 320,000 Da, such as a molecular weight between 102,000 Da and 310,000 Da, such as a molecular weight between 102,000 Da and 300,000 Da, such as a molecular weight between 102,000 Da and 275,000 Da, or such as a molecular weight between 102,000 Da and 250,000 Da. All end-points are included in the above ranges.

In one or more embodiments according to any aspect, the mucoadhesive polymer has a molecular weight between, such as between 110,000 Da and 340,000 Da, such as between 110,000 Da and 330,000 Da, such as between 110,000 Da and 325,000 Da, such as between 110,000 Da and 320,000 Da, such as a molecular weight between 110,000 Da and 310,000 Da, such as a molecular weight between 110,000 Da and 300,000 Da, such as a molecular weight between 110,000 Da and 275,000 Da, or such as a molecular weight between 110,000 Da and 250,000 Da. All end-points are included in the above ranges.

In one or more embodiments according to any aspect, the mucoadhesive polymer does not have a molecular weight of 150,000 Da, such as between 149,000 Da and 151,000 Da. All end-points are included in the above ranges.

In one or more embodiments according to any aspect, the mucoadhesive polymer has a molecular weight between 101,000 Da and 149,000 Da or between 151,000 Da and 350,000 Da, such as between 102,000 Da and 149,000 Da or between 151,000 Da and 350,000 Da, such as between 105,000 Da and 149,000 Da or between 151,000 Da and 350,000 Da, or such as between 110,000 Da and 149,000 Da or between 151,000 Da and 350,000 Da. All end-points are included in the above ranges.

In one or more embodiments according to any aspect, the mucoadhesive polymer has a molecular weight between 101,000 Da and 149,000 Da or between 151,000 and 340,000 Da, such as between 101,000 Da and 149,000 Da or between 151,000 and 330,000 Da, such as between 101,000 Da and 149,000 Da or between 151,000 and 330,000 Da, such as between 101,000 Da and 149,000 Da or between 151,000 and 320,000 Da, such as between 101,000 Da and 149,000 Da or between 151,000 and 310,000 Da, such as between 101,000 Da and 149,000 Da or between 151,000 and 300,000 Da, such as between 101,000 Da and 149,000 Da or between 151,000 and 275,000 Da, or such as between 101,000 Da and 149,000 Da or between 151,000 and 250,000 Da. All end-points are included in the above ranges.

In one or more embodiments according to any aspect, the mucoadhesive polymer does not have a molecular weight between 251,000 Da and 252,000 Da, such as between 250,000 Da and 253,000 Da. All end-points are included in the above ranges.

In one or more embodiments according to any aspect, the mucoadhesive polymer has a molecular weight between 101,000 Da and 149,000 Da or between 151,000 and 251,000 Da or between 252,000 Da and 350,000 Da, such as between 110,000 Da and 149,000 Da or between 151,000 and 251,000 Da or between 252,000 Da and 350,000 Da, such as between 120,000 Da and 149,000 Da or between 151,000 and 251,000 Da or between 252,000 Da and 350,000 Da, such as between 125,000 Da and 149,000 Da or between 151,000 and 251,000 Da or between 252,000 Da and 350,000 Da. All end-points are included in the above ranges.

In one or more embodiments according to any aspect, the mucoadhesive polymer has a molecular weight between 101,000 Da and 149,000 Da or between 151,000 and 251,000 Da or between 252,000 Da and 340,000 Da, such as between 101,000 Da and 149,000 Da or between 151,000 and 251,000 Da or between 252,000 Da and 330,000 Da, such as between 101,000 Da and 149,000 Da or between 151,000 and 251,000 Da or between 252,000 Da and 320,000 Da, such as between 101,000 Da and 149,000 Da or between 151,000 and 251,000 Da or between 252,000 Da and 310,000 Da, such as between 101,000 Da and 149,000 Da or between 151,000 and 251,000 Da or between 252,000 Da and 300,000 Da, such as between 101,000 Da and 149,000 Da or between 151,000 and 251,000 Da or between 252,000 Da and 275,000 Da, such as between 101,000 Da and 149,000 Da or between 151,000 and 251,000 Da. All end-points are included in the above ranges.

In one or more embodiments according to any aspect, the mucoadhesive polymer is a peptide molecule of a length of 469 to 4,661 amino acids, which are linked via amide bonds. When the mucoadhesive polymer comprises amino acids, any amino acid may be included as long as at least 50% of the amino acids carry a basic group, or as long as at least 50% of the amino acids carry a hydrophobic group as appropriate, or as long as at least 50% of the amino acids carry thiol groups, or a combination of these three (basic, hydrophobic, and thiol). The mucoadhesive polymer is not limited to naturally occurring amino acids, but it is preferred that the amino acids are non-toxic and tolerated by the subject. It is preferred that the mucoadhesive polymer does not comprise D-amino acids but that any amino acid contained in the mucoadhesive polymer is an L-amino acid.

In general, the following amino acids are considered basic: arginine, lysine, histidine, ornithine, and β-alanine, and in an embodiment the mucoadhesive polymer is a polypeptide of amino acids, wherein at least 50% of the amino acids are selected from the list consisting of arginine, lysine, histidine, ornithine, and β-alanine. The remaining amino acids may be selected from any amino acids, e.g. any of the 20 amino acids defined from the genetic code, but in particular glycine, serine, threonine, asparagine, and glutamine. Specific embodiments of the mucoadhesive polymer comprise poly-lysine, poly-orthinine, and/or poly-arginine. The advantage of using basic amino acids is that they have a good solubility in aqueous solutions.

In one or more embodiments according to any aspect, the mucoadhesive polymer is a peptide molecule of a length of 469 to 4,661 amino acids wherein at least 50% of the amino acids carry a hydrophobic group, which amino acids are selected from the list consisting of: alanine, methionine, cysteine, phenylalanine, leucine, valine, and isoleucine. The remaining amino acids may be selected from the list consisting of: glycine, serine, threonine, asparagine, and glutamine, or the remaining amino acids may be selected from any amino acids, e.g. any of the 20 amino acids defined from the genetic code.

In one or more embodiments according to any aspect, the mucoadhesive polymer comprises amino acids, wherein at least 50% of the amino acids are selected from the group consisting of arginine, lysine, histidine, ornithine, and β-alanine, or 50% of the amino acids carries a hydrophobic group, and are selected from the group consisting of alanine, methionine, cysteine, phenylalanine, leucine, valine, and isoleucine.

In one or more embodiments according to any aspect, the mucoadhesive polymer is a peptide molecule, which are linked via amide bonds, wherein at least 50% of the amino acids are selected from the list consisting of arginine, lysine, histidine, ornithine, and β-alanine. In another embodiment, at least 60% of the amino acids are selected from the list consisting of arginine, lysine, histidine, ornithine, and β-alanine. In yet another embodiment, at least 70% of the amino acids are selected from the list consisting of arginine, lysine, histidine, ornithine, and β-alanine.

In one or more embodiments according to any aspect, the mucoadhesive polymer is a peptide molecule, which are linked via amide bonds, wherein at least 50% of the amino acids are lysine. In another embodiment, at least 60% of the amino acids are lysine. In yet another embodiment, at least 70% of the amino acids are lysine.

In one or more embodiments according to any aspect, the mucoadhesive polymer comprises amino acids being L-lysine. In one or more embodiments according to any aspect, the mucoadhesive polymer is poly-L-lysine (PLL).

It is advantageous to use amino acids or hydrophobic amino acids since they are biodegradable. The protein-peptide interactions between the mucus proteins and the polymer may enhance mucoadhesion. Furthermore, the amino acid polymers may be produced recombinant using bacteria or synthetically.

In one or more embodiments according to any aspect, the mucoadhesive polymer comprises both sugar monomers, e.g. C6 sugar monomers, and amino acids, wherein at least 50% of the monomers are basic, e.g. carry an amino group, or at least 50% of the monomers are hydrophobic, e.g. carry a hydrophobic group.

In one or more embodiments according to any aspect, the mucoadhesive polymer consists of 616 to 2,054 monomer units linked to each other via ether bonds, ester bonds, amide bonds, or combinations hereof, such as 800 to 2,054 monomer units, such as 800 to 1,800 monomer units, such as 1,000 to 1,800 monomer units, such as 1,200 to 1,600 monomer units linked to each other via ether bonds, ester bonds, amide bonds, or combinations hereof. The size of the mucoadhesive polymer is very important as it ensures that the polymer forms a tight cross-linking network giving an effective barrier at the interface of the mucus layer or inside the pores of the mucus layer. By tight is meant e.g., impermeable to microorganism or sperm cells.

In one or more embodiments according to any aspect, the mucoadhesive polymer is selected from polymers with a low molecular weight, which should have a degree of polymerisation (DP) providing a molecular weight in the range of 90 to around 350 kDa, which ensures that the mucoadhesive polymer forms stable complexes with the mucus.

The size or the polymer ensures that the polymer is soluble in the conditions used, and that the polymer can diffuse through the pores of mucus and form a thick and tight barrier or only diffuse slightly into the mucus layer hereby forming a tight cross-linking network at the interface of the mucus layer.

The mucoadhesive polymer should be stable in the environment of the targeted mucosa, which is low pH in the female abdomen. The pH range where the mucoadhesive polymer is stable is therefore in the range of 1-8. Dependent on the pH environment, different types and sizes of polymers may be used.

In one or more embodiments according to any aspect, pH of the vaginal contraceptive composition is between 2.0 and 7.0, such as between 2.5 and 6.5, such as between 3.0 and 6.0. All end-points are included in the above range.

By a pH of the composition between 2.0 and 7.0 is meant that the pH, if measured, is between the two values and that the mucoadhesive polymer is stable in this range. A lower pH of the composition is preferable if the compositions are to be applied to the female abdomen, especially to the female vagina, where pH value is ranging from 3 to 5.

To maintain pH at a specific level in the composition a buffer or other pH stabilising solution may be added to the composition, such that the one or more active ingredients are administered in a physiologically acceptable buffer solution, which ensures that the one or more active ingredients are soluble in the conditions where it is used, that the one or more active ingredients are evenly distributed in the target area, that the active ingredients are correctly charged, and that the natural pH value of the female abdomen, especially to the female vagina is maintained when the composition is applied.

Hence, in one or more embodiments according to any aspect, the composition comprising one or more active ingredients, at least one formulation compound selected from a physiological acceptable gelling agent or a physiological acceptable carrier, and a physiologically acceptable buffer solution. In one or more embodiments according to any aspect, the one or more active ingredients is administered in at least one physiologically acceptable gelling agent and at least one physiologically acceptable buffer solution. In one or more embodiments according to any aspect, the composition comprising one or more active ingredients, a physiological acceptable gelling agent, and a physiologically acceptable buffer solution. In one or more embodiments according to any aspect, the one or more active ingredients is administered in at least one physiologically acceptable carrier and at least one physiologically acceptable buffer solution. In one or more embodiments according to any aspect, the composition comprising one or more active ingredients, a physiological acceptable carrier, and a physiologically acceptable buffer solution. In one or more embodiments according to any aspect, the composition comprising one or more active ingredients, a physiological acceptable gelling agent, a physiologically acceptable carrier, and a physiologically acceptable buffer solution.

By physiological acceptable buffer solution is meant a non-toxic buffer solution, which in an effective concentration to maintain pH, is neither chemically nor physically toxic to a human and/or animal organism.

In one or more embodiments according to an aspect, the physiological acceptable buffer solution is selected from a lactic acid solution, a citric acid solution, an acetic acid solution, succinic acid solution, malic acid solution, tartaric acid and potassium bitartrate combined with citric acid, or combinations hereof.

In one or more embodiments according to any aspect, the buffer solution is in a concentration from 1 mM to 350 mM, such as a concentration from 1 mM to 325 mM, such as a concentration from 1 mM to 300 mM, such as a concentration from 2 mM to 300 mM, such as a concentration from 3 mM to 300 mM, such as a concentration from 4 mM to 300 mM, such as a concentration from 5 mM to 300 mM, such as a concentration from 10 mM to 300 mM, such as a concentration from 15 mM to 300 mM, such as a concentration from 20 mM to 300 mM, such as a concentration from 20 mM to 250 mM, or such as a concentration from 20 mM to 200 mM. All end-points are included in the above ranges.

To maintain a stable composition over a larger period, e.g. for maximizing the shelf life of such composition, a preservative may be added to the composition, such that the one or more active ingredients are administered in with physiologically acceptable preservative, which ensures an acceptable shelf life of the final compound.

A preservative is a substance or a chemical that is added to products such as food products, beverages, pharmaceutical drugs, paints, biological samples, cosmetics, wood, and many other products to prevent decomposition by microbial growth or by undesirable chemical changes. In general, preservation is implemented in two modes, chemical and physical. Chemical preservation entails adding chemical compounds to the product. Physical preservation entails processes such as refrigeration or drying.

Hence, in one or more embodiments according to any aspect, the composition comprising one or more active ingredients, at least one formulation compound selected from a physiological acceptable gelling agent or a physiological acceptable carrier, and at least one physiologically acceptable preservative. In one or more embodiments according to any aspect, the one or more active ingredients is administered in at least one physiologically acceptable gelling agent and at least one physiologically acceptable preservative. In one or more embodiments according to any aspect, the composition comprising one or more active ingredients, a physiological acceptable gelling agent, and a physiologically acceptable preservative. In one or more embodiments according to any aspect, the one or more active ingredients is administered in at least one physiologically acceptable carrier and at least one physiologically acceptable preservative. In one or more embodiments according to any aspect, the composition comprising one or more active ingredients, a physiological acceptable carrier, and a physiologically acceptable preservative. In one or more embodiments according to any aspect, the composition comprising one or more active ingredients, a physiological acceptable gelling agent, a physiologically acceptable carrier, and a physiologically acceptable preservative. In one or more embodiments according to any aspect, the composition comprises both a physiologically acceptable buffering solution and a physiologically acceptable preservative in combinations with one or more active ingredients and at least one formulation compound selected from a physiological acceptable gelling agent or a physiological acceptable carrier.

By physiological acceptable preservative is meant a non-toxic preservative, which in an effective concentration, is neither chemically nor physically toxic to a human and/or animal organism.

In one or more embodiments according to an aspect, the physiological acceptable preservative is a chemical preservative.

In one or more embodiments according to an aspect, the physiological acceptable preservative is selected from sorbic acid, sodium sorbate, sorbates, benzoic acid, benzoates, parabens, sulfur dioxide, sulphites, nitrites, nitrates, lactic acid, propionic acid, propionates, citric acid, formic acid, dehydroacetic acid, phenethyl alcohol, caprylyl glycol, or combinations hereof.

In one or more embodiments according to any aspect, the preservative is in a concentration of between 0.05 wt. % and 5.0 wt. % of the total weight of the vaginal contraceptive composition, such as between 0.05 wt. % and 3.0 wt. %, such as between 0.05 wt. % and 3.0 wt. %, such as between 0.05 wt. % and 2.5 wt. %. such as between 0.08 wt. % and 2.5 wt. %, such as between 0.10 wt. % and 2.5 wt. %, such as between 0.10 wt. % and 2.3 wt. %, such as between 0.10 wt. % and 2.2 wt. %, or such as between 0.10 wt. % and 2.0 wt. % of the total weight of the vaginal contraceptive composition.

The diffusion of the mucoadhesive polymer occurs when the mucoadhesive polymer adheres to the mucus. The use of the mucoadhesive polymers in therapy is possible due to its degree of polymerisation and degree of acetylation, which gives good mucoadhesion. This allows the polymer to diffuse into the mucus and temporarily block the pores of the mucus. This occurs due to a temporary cross-linking effect of the mucus, which is controlled by the normal turnover of mucus and the biodegradability of the mucoadhesive polymer. The effective cross-linking time can therefore be adjusted by subjecting the mucus to different concentrations of the mucoadhesive polymer such as for example a concentration from 1 mg/mL to 150 mg/mL, such as a concentration from 1 mg/mL to 100 mg/mL, such as a concentration from 1 mg/mL to 75 mg/mL, such as a concentration from 1 mg/mL to 50 mg/mL, such as a concentration from 1 mg/mL to 25 mg/mL, such as a concentration in the range of 5 mg/mL. All end-points are included in the above ranges.

In one or more embodiments according to any aspect, the mucoadhesive polymer is in a concentration of between 0.05 wt. % and 15.0 wt. % of the total weight of the vaginal contraceptive composition, such as between 0.05 wt. % and 10.0 wt. %, such as between 0.5 wt. % and 10.0 wt. %, such as between 1.0 wt. % and 10.0 wt. %, such as between 2.5 wt. % and 10.0 wt. %. such as between 5.0 wt. % and 10.0 wt. %, such as between 0.05 wt. % and 10.0 wt. %, such as between 0.05 wt. % and 8.0 wt. %, such as between 0.05 wt. % and 6.0 wt. %, such as between 0.05 wt. % and 4.0 wt. % of the total weight of the vaginal contraceptive composition.

By between 0.05 wt. % and 10.0 wt. % is meant that between 0.05% and 10.0% of the total weight of the vaginal contraceptive composition is from the mucoadhesive polymer. All end-points are included in the above ranges.

The mucoadhesive polymer will, due to its adhesive properties and size, penetrate the mucus and diffuse into the surface of the mucus to form a thick layer. The mucoadhesive polymer will then complex to the mucus and thereby block the pores of the network, providing a reinforced-barrier property to the mucus. When the mucus is reinforced, it is impermeable to particles, and prevents passage e.g. externally induced liquids, particles and cells, such as spermatozoa. The complex formed in the mucus could be targeted against a certain size of cells, and thereby be impermeable to virions (or viruses) of the range of 20 to 30 nm in size, mycoplasma in the range of 0.3 microns, bacteria in the range of 0.5 to 5 microns, or spermatozoa in the range of 3 microns.

The composition of the invention comprising a mucoadhesive polymer and at least one formulation compound functions as a contraceptive agent, since the treated mucus will be temporarily impermeable to spermatozoa. The contraceptive effect in connection with the present invention means a reversible and temporary prevention of pregnancy due a non-surgical and hormone-free barrier effect achieved by a single use, meaning that the contraceptive effect is achieved by one application, and does not require a concentration to be developed over a period of time, as it does with e.g. hormone pills such as a combined oral contraceptive pill (often referred to as the birth control pill or colloquially as “the pill”).

By temporary effect is implied that, when applied to the mucus, the effect of the mucoadhesive polymer is reversible. The rate at which the reversion will occur is determined by the amount of polymer diffused into the mucus and by the biological turnover of the mucus itself.

In another aspect, the invention is a kit of parts, which comprises the vaginal contraceptive composition comprising the mucoadhesive polymer and the at least one formulation compound selected from a physiological acceptable gelling agent or a physiological acceptable carrier, e.g. a contraceptive composition as described above, and an applicator. In one embodiment, the applicator is a delivery device utilising a method where the applicator comprising the vaginal contraceptive composition in the form of a gel, which is inserted in the vagina via a syringe or by introduction of a soft-gel capsule that dissolves in the vagina, releasing the gel. The gel is deployed from the applicator and applied to the cervical mucus; hereby the mucus is cross-linked by the mucoadhesive polymer. In an embodiment, the applicator is a container, which contains the vaginal contraceptive composition, and which can be emptied by an emptying mechanism.

The composition comprising the mucoadhesive polymer and at least one formulation compound selected from a physiological acceptable gelling agent or a physiological acceptable carrier may be part of a kit further comprising an applicator. The applicator may be used to apply the composition to the surface of e.g. the cervix.

In one embodiment, the applicator is a syringe. In another embodiment, the applicator is a soft-gel capsule.

In a further embodiment, the kit comprises the vaginal contraceptive composition, an applicator, and instructions for use.

A vaginal contraceptive composition according to any embodiment may be used at any time of day, ahead of intercourse. Preferably, between 24 hours to 30 seconds before intercourse. The vaginal contraceptive composition may be used with the intention to prevent pregnancy and it may be administered in an amount of between 1 to 5 mL by vaginal administration, using either a syringe or a soft-gel capsule. The vaginal contraceptive composition is to be inserted vaginally, either using fingers or an applicator.

A vaginal contraceptive composition according to any embodiment may be produced by:
The active ingredient powder, such as chitosan powder, is mixed with a gelling agent or carrier, such as hydroxyethylcellulose Natrosol 250 HX (HEC);
Optionally, a buffer composition is added, for instance composed of lactic acid and succinic acid;
Then optionally, water can be added to bring the components in suspension and continuously stirred with a vortex mixer to give a homogeneous gel;
Optionally, NaOH and/or HCl solutions can be added dropwise to adjust the pH of the solution;
The semi-solid formulation can optionally be centrifuged to remove air trapped in the gel; and
A preservative, for instance benzoic acid, can be added to the gel formulation.

The present invention is further illustrated by the following examples, which are not to be construed as limiting the scope of protection. The features disclosed in the foregoing description and in the following examples may, both separately or in any combination thereof, be material for realizing the invention in diverse forms thereof.

Various examples are described hereinafter with reference to the figures. It should also be noted that the figures are only intended to facilitate the description of the examples. They are not intended as an exhaustive description of the claimed invention or as a limitation on the scope of the claimed invention. In addition, an illustrated example needs not have all the aspects or advantages shown. An aspect or an advantage described in conjunction with a particular example is not necessarily limited to that example and can be practiced in any other examples even if not so illustrated, or if not so explicitly described.

### EXAMPLES

In here it is shown that the hydrogels of porcine gastric mucin and the mucus layer produced by colonic cell lines could be altered by low molar mass chitosan (below 2,000 Da), which would reinforce the barrier properties and slows the diffusion of dextran polymers and a subunit of the cholera toxin through the hydrogels.

Although all mucus gels share similar characteristics, they also differ by a number of ways. This includes mucin contraction (e.g. pore size), mucin concentration and type of associated protein and lipids, and salt concentration. Additionally, they also differ in environmental factors such as pH, exposure to shear stress, exposure to bacteria, and different turnover rates. Given these differences, it is therefore not obvious that a treatment designed for porcine mucin hydrogels would also work in reinforcing the barrier properties of cervical mucus.

Materials and Methods

Chitosan Preparation.

All chitosans were dissolved in a 100 mM or a 32.5 mM lactic acid solution at a concentration of 0.5% (w/v chitosan) and adjusted to pH 5.5 (pH±0.02) by the use of 0.1 or 1 M hydrochloric acid (HCl, Merck KgaA, Germany), and 0.1 or 1 M sodium hydroxide (NaOH, CPAchem Ltd., Bulgaria) or 50% NaOH (Sigma-Aldrich, USA).

TABLE 1
Characteristics of the chitosans used.

| Chitosan | | |
|---|---|---|
| (Identification name) | DDA (%) | Mw (kDa) |
| CO | NC | 1.4 (±0.7%) |
| Z49 | 98.9 | 7.1 (±0.4%) |
| Z56 | 93.7 | 18.9 (±0.3%) |
| 95/50 | 98.1 | 102.3 (±0.4) |
| CsH | 94.2 | 131.8 (±0.1) |
| 95/100 | 95.0 | 150.0 (±0.2%) |
| 95/100_2 | 97.7 | 175.6 (±0.4%) |
| Z43 | 96.9 | 251.8 (±0.2%) |
| 95/1000 | 94.7 | 290.9 (±0.4%) |
| Z47 | 94.4 | 315.9 (±0.2%) |

DDA: Deacetylation degree; Mw: molecular weight; NC: Not calculable.

Semen Assessment

Semen samples of patients and volunteers collected at the Andrology, Sexual Medicine, Transmedicine, clinic (ANOVA, Karolinska University Hospital, Sweden) were run through standard semen kinematic analyses and sperm penetration assays no later than 3 h after collection. Data were obtained for the collection time, abstinence time, and semen volume.

Semen was gently liquefied for 30 min on a rocker in an incubation chamber heated up to 37° C., after complete collection of ejaculates by masturbation. The viscosity of semen was determined visually and by pipetting. Semen was microscopically analysed by pipetting 6 μl of specimen in pre-warmed Leja® (Netherland) counting chamber slides (20 micron). Subsequently, specimens were assessed via clinical ECLIPSE 50i microscope (Nikon Instruments, Japan) equipped with stage heater MS 100 (37° C., Linkam Scientific Instruments, UK), 10× objective (Ph1) and 0.5× charge coupled device camera UI-1540LE-M-HQ (IDS Imaging Development Systems GmbH, Germany) with total magnification of 5×. The system was connected to the Computer Aided Semen Analysis software QualiSperm (v3.0.9.486, AKYmed, Switzerland). Beside the sperm concentration [106/ml], the progressive motility [%], motility [%], immotility [%], velocity [μm/s], sperm size [μm2], and number of cells were measured. Semen samples meeting the following criteria were included in this study: volume of >1.5 ml, concentration of >15×106/ml and >40% progressive motility. These criteria reflect reference limits of the World Health Organization (WHO) (“WHO Laboratory Manual for the Examination and Processing of Human Semen” 2010) and values for normal spermatozoa as stated in Björndahl (Lars Björndahl. 2011. “What Is Normal Semen Quality? On the Use and Abuse of Reference Limits for the Interpretation of Semen Analysis Results.” Human Fertility 14 (3): 179-86). In each run, ten values were generated by assessing five fields in two chambers.

Cervical Mucus Assessment

Ovulatory cervical mucus (CVM) (the CVM with highest penetrability) was collected from healthy donors at the Karolinska University Hospital. Donors were not using hormonal contraceptives, were between 18 and 30 years old, had a BMI of 19-25, were non-smokers, and were neither under medication nor affected by chronic diseases. Before the collection of CVM, the hormonal status of each healthy, regularly cycling volunteer was examined by blood tests at the Karolinska University Laboratory. At the moment of donation, the woman's level of FSH, LH, and estradiol was analysed to prove the prevalence of ovulation. Mucus was collected at the external orifice using the endometrial catheters Gynebiops standard CH9 (GYNEAS, France) and Pipelle de Cornier for endometrial biopsy (PRODiMED, France).

Phase Contrast Microscopy of Spermatozoa Penetration

After the liquefaction of semen, and assessment of semen and CVM, 100 μl of buffer only and chitosan in buffer were filled in glass vials (ND9, 1.5 ml, VWR, USA) closed by caps with septum (55° shore, Teknolab Sorbent AB, Sweden), and pre-heated at 37° C. An aliquot of ovulatory CVM was aspirated into two customized capillaries and the capillaries were sealed at the broken end as described above. The septum of the cap was penetrated by the sealed end of the capillaries filled with CVM. The capillaries, embedded through the caps, were first placed in glass vials containing chitosan solutions for 30 min at 37° C. (5 mm deep into the solution). Then the capillaries were transferred to a glass vial containing 100 μl of semen for penetration of spermatozoa for 30 min at 37° C. (5 mm deep into the solution). For one control experiments, the same process was repeated but replacing chitosan solution with only buffer solution. For another control experiment, the same process was repeated but directly dipping the capillaries filled with ovulatory CVM in sperm. After incubation, the capillaries were placed on a customized microscopic glass slide marked with the distances 0.5, 1, 2, 3, 4, and 5 cm, positioned on a pre-warmed (37° C.) stage heater DC 95 (Linkam Scientific Instruments, UK), and observed by microscopy.

Videos were recorded at the distances marked on the glass slide, including the beginning of the capillary (0.1 cm), by the use of the Eclipse Ci phase contrast microscope (Nikon, Japan) equipped with UI-3240LE-C-HQ camera (IDS Imaging Development Systems, Germany). A magnification of 10× was used for the objective (Ph1) and camera, generating a total magnification of 100×. The recorded microscopic field was 0.21×0.27 mm, equivalent to 0.0567 mm2. A resolution of 1280×1024 pixel were used to record videos of three fields at each distance with 30 pictures per second. The recording started at the upper, outer surface of the capillary, followed by focusing through the capillary until reaching the lower surface, resulting in a 3-D scan through the capillary. Spermatozoa were counted in the volume (0.017 mm³). The assay was conducted in triplicate using semen of different volunteers.

Pipettable semen with a volume of ≥1.5 ml, a sperm counts of ≥15 mill spermatozoa/ml and a progressive motility of at least 40% were used. Only the most sperm-epenetrative, transparent phases of ovulatory cervical mucus was used. The ovulatory quality of the mucus was verified by the ferning of mucin under light microscope and the level of luteinizing hormone, follicle-stimulating hormone and estradiol. Only mucus scored with at least 8 points out of 12, accordingly to the Insler scoring, were used. Acceptable pH range for the mucus was between 6.5 and 8.5.

In Vivo Sperm Penetration in the Ewe Following Chitosan Formulation Treatment

In vivo studies are performed on Ile de France ewes. The oestrus is synchronized with progesten intravaginal sponges (Fluorogestone acetate 30 mg, Sanofi Animal Health) inserted 14 days before artificial insemination (AI). Females are maintained in field conditions for 2 weeks, sponges are retrieved and 400 IU of pregnant mare's serum gonadotropin (PMSG) is injected to each ewe inducing optimized reproductive condition.

Artificial insemination is performed 72 hours following sponge removal without detection of oestrus.

The study is performed with ejaculates produced by Lacaune rams. Collected Semen is filtered and quality is assessed by phase contrast microscopy (BH2-RFCA microscope, Olympus). Sperm concentration is estimated using an absorbance spectrophotometer colorimeter 254 (Ciba Corning). Semen is used when the mass motility score is over 4 and the sperm concentration is at least 3×109 sperm/mL. The semen collected is incubated with of R18 fluorochrome (0.01% v/v) (Octadecyl Rhodamine B chloride, 0-246, invitrogen) labelling over the surface of spermatozoon and flagella and MitoTracker Green FM (0.01% v/v) labelling mitochondria of spermatozoa. Semenal fluids and dyes are washed out by centrifugation (800 g, 40 min, 37° C.) in discontinuous percoll gradient (45% v/v and 90% v/v). The semen is diluted to a final concentration of 1×10⁹ sperm/mL in warm (37° C.) skimmed milk (11% w/v). 1 mL syringes of final semen solution is prepared for artificial insemination of each ewe.

Examination of vagina and cervix entrance is performed with the Cellvizio fluorescence endo-microscope prior formulation and sperm insemination. A control is performed with a confocal probe to record eventual far red fluorescence emission and the level of auto-fluorescence.

One hour after chitosan formulation application the ewes are artificially inseminated. Four hours after AI the animals are sacrificed and confocal fluorescence endomicroscopy (Cellvizio) is performed in the following areas: vagina, posterior cervix, anterior cervix, uterus, uterus and oviduct junction and the oviducts. Sperm and chitosan quantification are assessed using the Image J software. Video sequences are recorded for the different regions of the genital tract. The quantitation of chitosan fluorescence intensity is analysed with the Cellvizio IC viewer and the total number of sperm per field is counted and subjected to motility analysis to obtain the percentage of motile sperm.

### Example 1

The impact of the chitosan on the barrier properties of human ovulatory cervical mucus was tested using formulation-containing chitosans of various molar masses. In this assay, human ovulatory cervical mucus is exposed to formulation containing the chitosans solubilized in lactic acid, the chitosan formulation is then removed and replaced by freshly collected human semen. By measuring sperm distribution through the capillary after 30 minutes and comparing to untreated mucus, one can highlight the ability of such chitosan formulation to reinforce the barrier properties of the mucus.

Each chitosan tested was characterized by size exclusion chromatography to determine their molecular weights.

### Results of Example 1

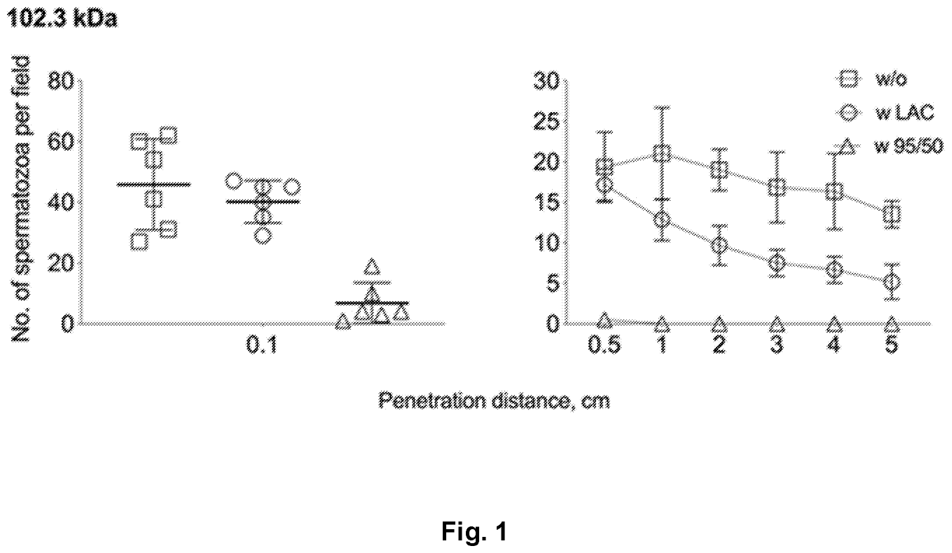

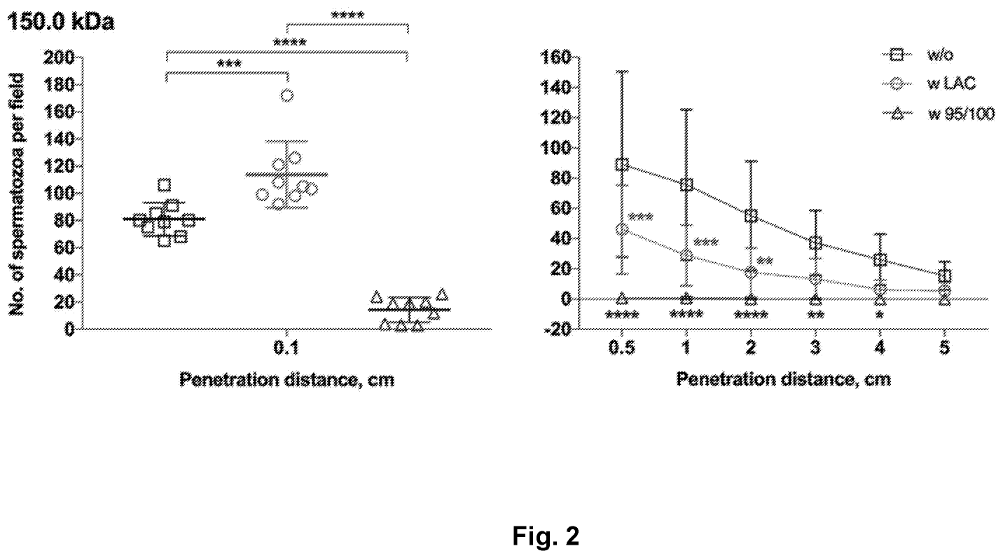

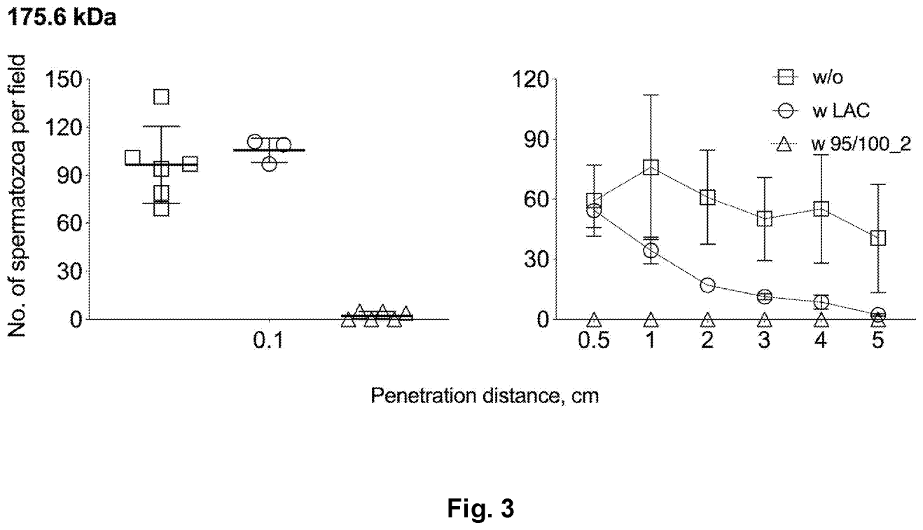

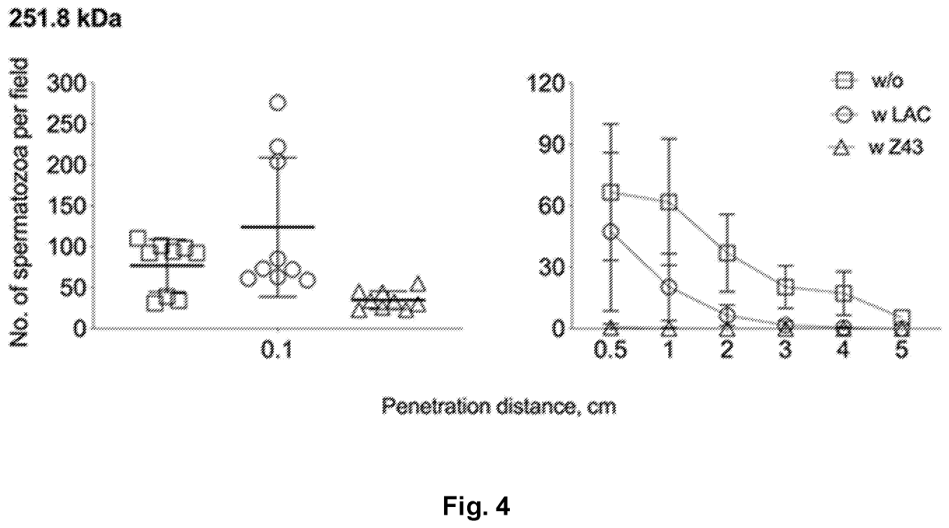

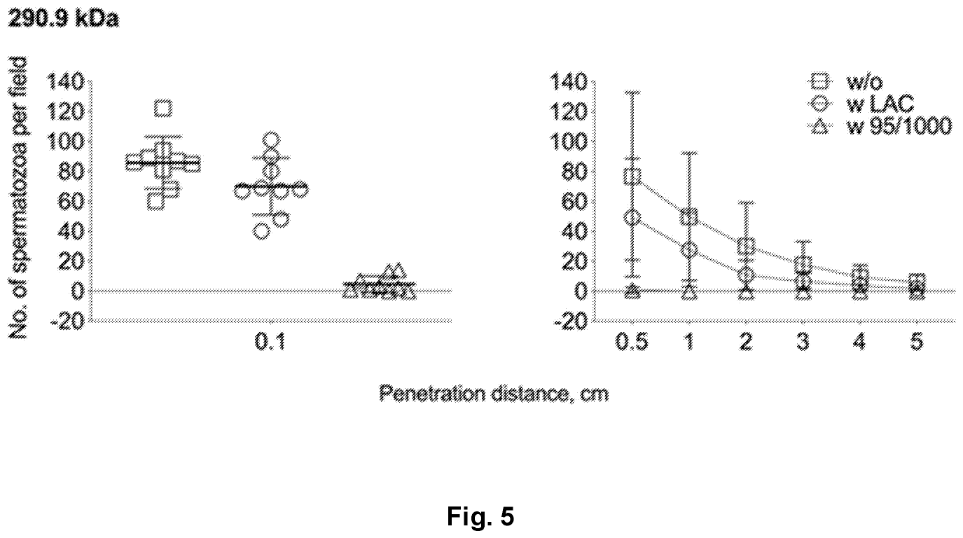

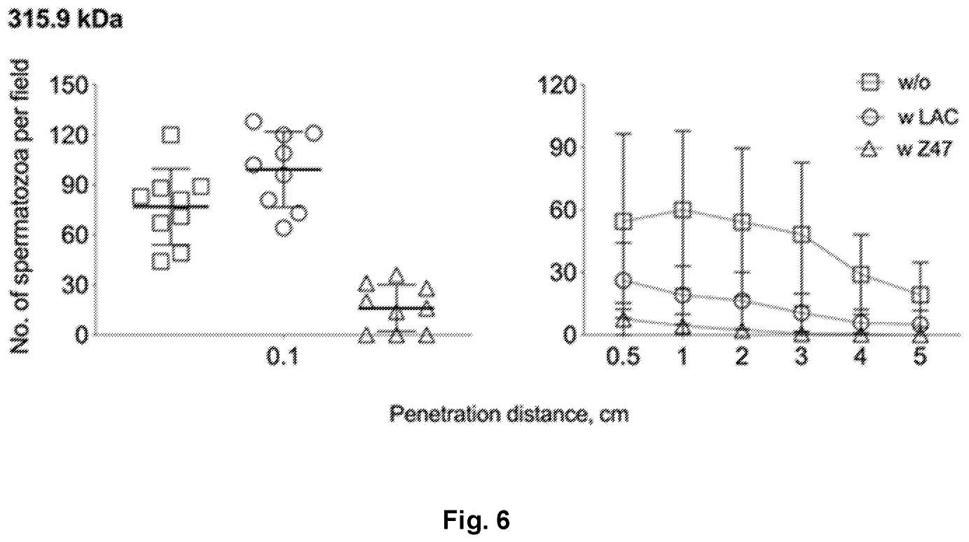

These results presented in FIGS. 1-6 show that the ovulatory cervical mucus allows significant number of sperm to penetrate the capillary tube for the 30 minutes exposure to the semen. Treatment of cervical mucus with lactic acid solution used to dissolve the chitosan did not have a strong effect on the sperm penetration. After exposure to the ovulatory cervical mucus to a chitosan solution for 30 minutes, the ovulatory cervical mucus was rendered poorly permeable to sperm. These results demonstrate that the treatment of ovulatory cervical mucus with chitosan measured to be 102.3, 150.0, 175.6, 251.8, 290.9, and 315.9 kDa all decreased significantly the ability of sperm to penetrate the ovulatory cervical mucus compared to either lactic acid-treated or non-treated ovulatory cervical mucus.

### Example 2

The barrier reinforcing effect of chitosan formulation was also confirmed in an in vivo model, with two chitosans of different sizes, CsH (131.8 kDa) and 95/50 (102.3 kDa). Similar to the sperm penetration assay of Example 1, ovulatory cervical mucus is exposed to the chitosan solution, and then the penetration of sperm is assessed. However, here the treatment is done in the animal, and the sperm penetration is through the reproductive tract of the animal, from the vaginal to the uterus. A combination of fluorescence labelling of sperm and fluorescence endo-microscopy techniques allow the detection of sperm through the reproductive tract after the chitosan treatment.

For the proper delivery of chitosan to the cervical canal, the chitosan formulation should contain excipients that increase the viscosity of the solution in order to increase the residency time of the formulation, and to avoid leakage. It is important that the excipients do not interact with the chitosan as to allow the full interaction with the mucus components. The selected gelling excipients should at least be known for their good biocompatibility, have no negative charges that could interact with positive changes of chitosan, and not have known interaction with chitosans. Hydroxyethycellulose was deemed a compatible thickening agent and were used in these studies.

### Results of Example 2

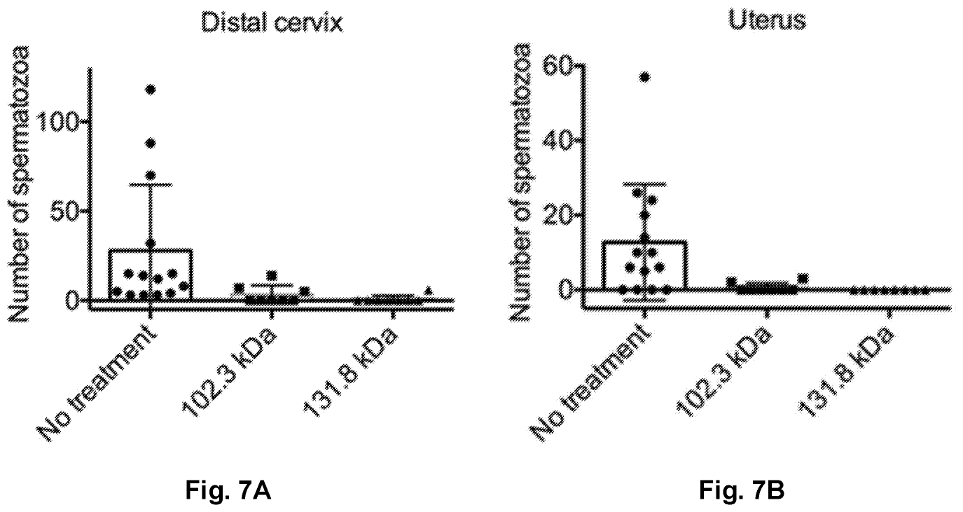

Control ewes, which were artificially inseminated while ovulating, but not previously treated with chitosan, had a number of sperm detected in their distal cervix and uterus. The number of sperm detected in the distal cervix and uterus was significantly reduced when the chitosan formulation was first placed in the ewe's vagina, one hour before artificial insemination. This experiment demonstrates the barrier reinforcing effect of formulation containing 102.3 kDa (95/50) and 131.8 kDa (CsH) chitosan in vivo. The results are shown in FIG. 7.

### Example 3

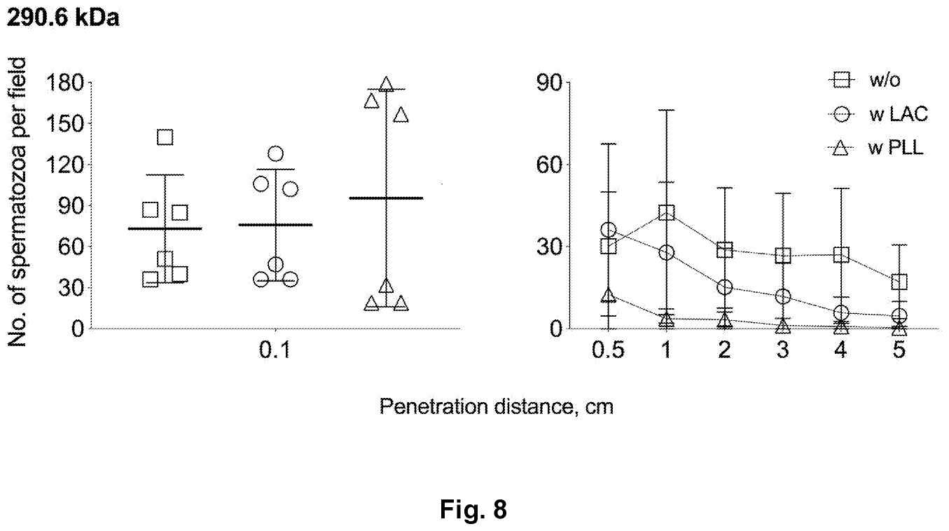

This example is to demonstrate mucus reinforcement by “amino acid monomers”. In the present example is tested the penetration of human sperm into human ovulatory cervical mucus first exposed to a solution of poly-L-lysine (PLL), dissolved at 5 mg/mL in lactic acid solution (32.5 mM lactic acid). The results are presented in the FIG. 8 showing sperm penetration assay performed on human ovulatory mucus. The sperm numbers were assessed 30 minutes after exposure to undiluted sperm.

Materials and methods used in example 3 are similar to the material and methods used in example 1 but performed by replacing chitosan with PLL, with a molecular weight ranging of 290.6 kDa as measured by viscometry.

### Results of Example 3

Less sperm was detected throughout the capillary filled with ovulatory cervical mucus pre-treated with the PLL solution than when treated with lactic acid solution alone or untreated. It can be deducted that the PLL compounds are able to reinforce the barrier properties of human ovulatory cervical mucus to sperm.

### Comparative Example 1

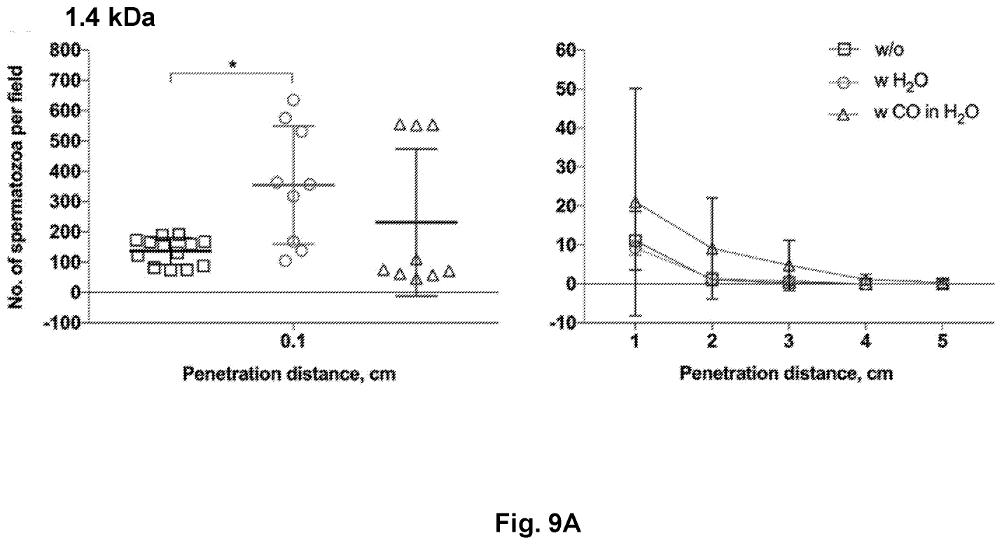

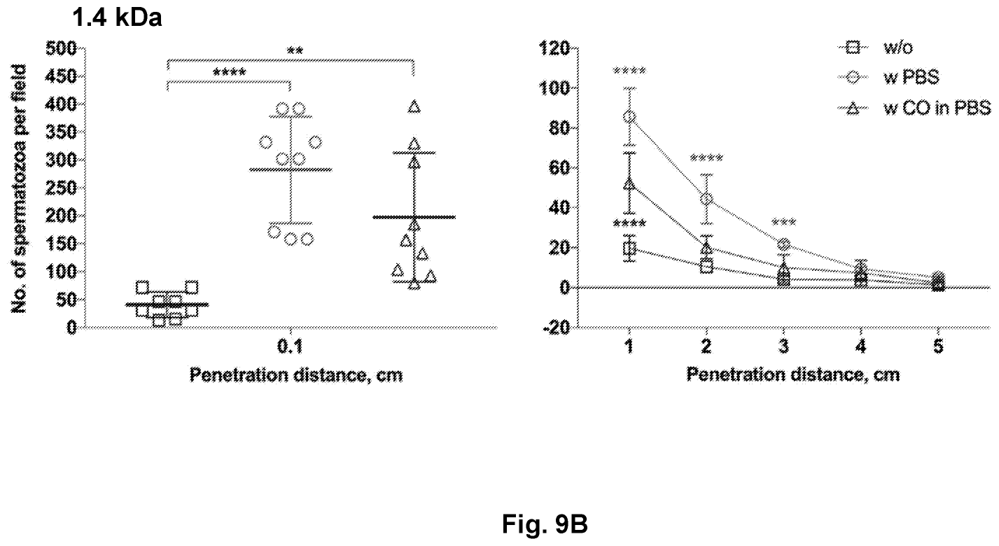

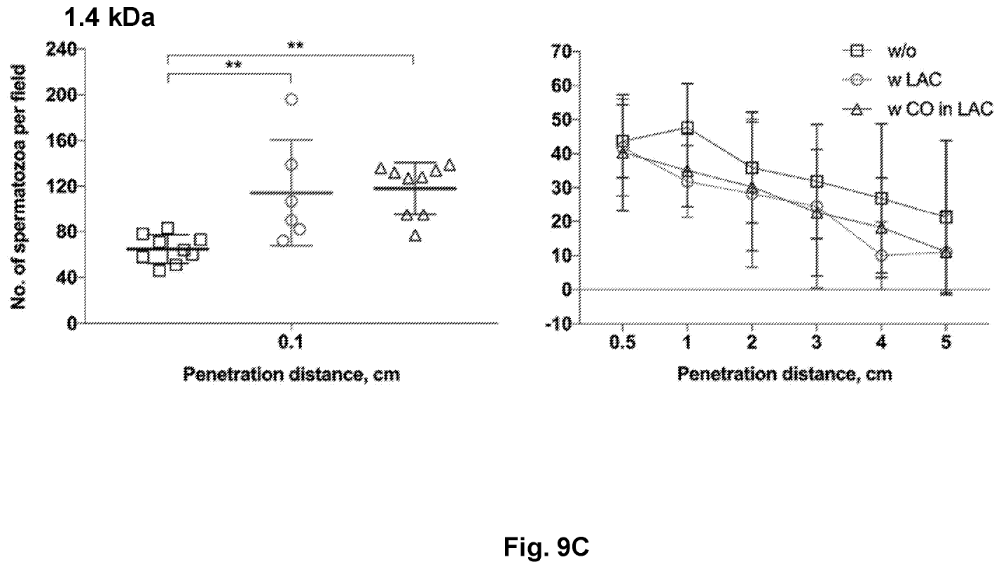

The impact of chitosan molar mass was evidenced by testing the diffusion of fluorescently labelled chitosans of various sizes through human ovulatory cervical mucus. Both chitosans of animal origin (extracted from the shells of crustaceans, CO) and for the fungal based chitosans (Z49, Z56). The impact of the molar mass of chitosan on the barrier properties of human ovulatory cervical mucus was tested using formulation-containing chitosans of various molar masses. By using the sperm penetration assays, human sperm, and ovulatory cervical mucus, it could be identified that smaller chitosan that were effective in reinforcing porcine gastric mucin hydrogels and mucus expressed by colonic cell lines, could not stop the penetration of human sperm through the mucus. This was clearly evidenced for the CO chitosan in several different buffer systems (FIG. 9), in a pH of 5.5 and lactic acid buffer.

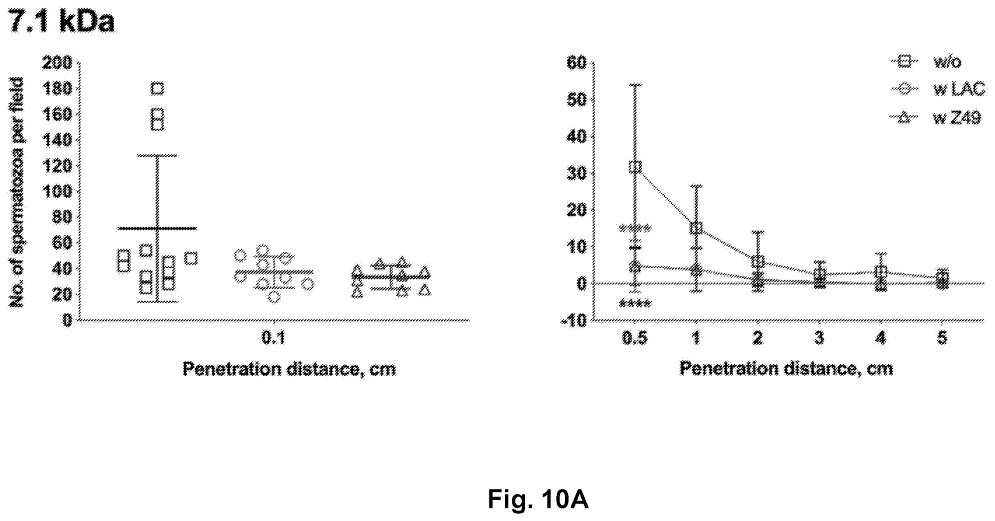

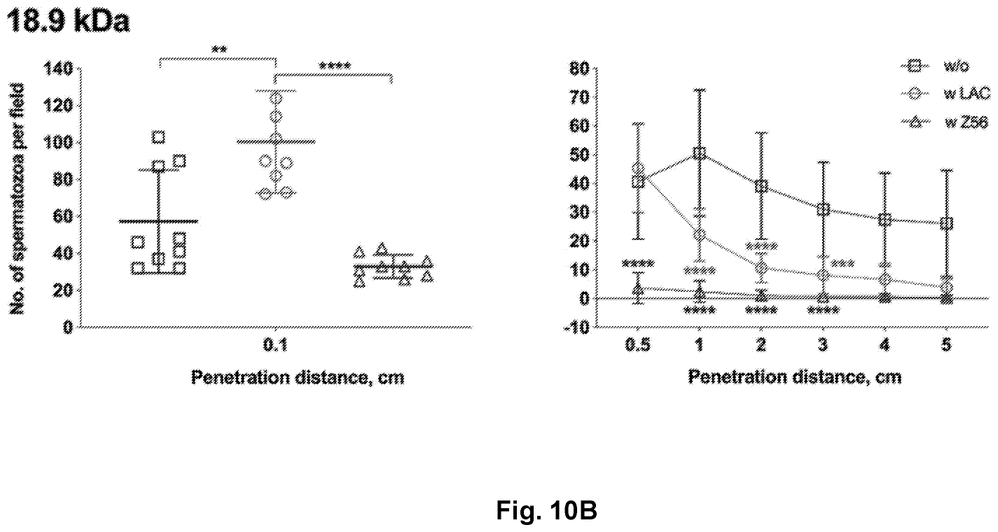

Similarly, it was shown that fungal chitosans of 7.1 and 18.9 kDa were also not effective at stopping sperm penetration (FIG. 10), Z49 and Z56 respectively.

The invention will hereafter be described by way of the following non-limiting items.
1. A vaginal contraceptive composition comprising one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.
2. The vaginal contraceptive composition according to item 1, wherein the composition comprises one or more active ingredients, a physiological acceptable gelling agent, and a physiologically acceptable carrier.
3. The vaginal contraceptive composition according to any preceding item, wherein the physiological acceptable carrier is in a concentration of between 5.0 wt. % and 99.0 wt. % of the total weight of the vaginal contraceptive composition, such as between 5.0 wt. % and 90.0 wt. %, such as between 5.0 wt. % and 80.0 wt. %, such as between 10.0 wt. % and 75.0 wt. %, such as between 10.0 wt. % and 70.0 wt. %. such as between 10.0 wt. % and 60.0 wt. %, such as between 10.0 wt. % and 50.0 wt. %, such as at least 5.0 wt. %, or such as at least 10.0 wt. % of the total weight of the vaginal contraceptive composition.
4. The vaginal contraceptive composition according to any preceding item, wherein the physiological acceptable gelling agent is in a concentration of between 0.05 wt. % and 50.0 wt. % of the total weight of the vaginal contraceptive composition, such as between 0.10 wt. % and 50.0 wt. %, such as between 0.10 wt. % and 40.0 wt. %, such as between 0.10 wt. % and 30.0 wt. %. such as between 0.10 wt. % and 20.0 wt. %, such as between 0.05 wt. % and 10.0 wt. %, such as between 0.10 wt. % and 10.0 wt. %, or such as between 0.50 wt. % and 10.0 wt. % of the total weight of the vaginal contraceptive composition
5. The vaginal contraceptive composition according to any preceding item, wherein the vaginal contraceptive composition has an osmolality of at least 50 mOsm/kg, such as at least 75 mOsm/kg, or such as at least 100 mOsm/kg.
6. The vaginal contraceptive composition according to any preceding item, wherein the vaginal contraceptive composition is not a foam.
7. The vaginal contraceptive composition according to any preceding item, wherein the mucoadhesive polymer has a molecular weight between 100,000 Da and 350,000 Da, such as between 101,000 Da and 350,000 Da.
8. The vaginal contraceptive composition according to any preceding item, wherein the mucoadhesive polymer has a molecular weight between 102,000 Da and 350,000 Da, such as between 102,000 Da and 325,000 Da.
9. The vaginal contraceptive composition according to any preceding item, wherein the mucoadhesive polymer has a molecular weight between 110,000 Da and 350,000 Da, such as between 110,000 Da and 325,000 Da.
10. The vaginal contraceptive composition according to any preceding item, wherein the mucoadhesive polymer has a molecular weight between 120,000 Da and 350,000 Da, such as between 120,000 Da and 325,000 Da.
11. The vaginal contraceptive composition according to any preceding item, wherein the mucoadhesive polymer has a molecular weight between 100,000 Da and 149,000 Da or between 151,000 and 350,000 Da, such as between 101,000 Da and 149,000 Da or between 151,000 and 350,000 Da, such as between 101,000 Da and 149,000 Da or between 151,000 and 325,000 Da, such as between 110,000 Da and 149,000 Da or between 151,000 and 325,000 Da, such as between 120,000 Da and 149,000 Da or between 151,000 and 325,000 Da, or such as between 125,000 Da and 149,000 Da or between 151,000 and 325,000 Da.
12. The vaginal contraceptive composition according to any preceding item, wherein said mucoadhesive polymer consists of a plurality of monomer units linked to each other via ether bonds.
13. The vaginal contraceptive composition according to any preceding item, wherein at least 55% of the monomer units comprise at least one amino group.
14. The vaginal contraceptive composition according to any preceding item, wherein at least 60% of the monomer units comprise at least one amino group.
15. The vaginal contraceptive composition according to any preceding item, wherein at least 65% of the monomer units comprise at least one amino group.
16. The vaginal contraceptive composition according to any preceding item, wherein at least 70% of the monomer units comprise at least one amino group.
17. The vaginal contraceptive composition according to any preceding item, wherein one or more of the at least one amino group are primary amines.
18. The vaginal contraceptive composition according to any preceding item, wherein the monomer units are selected from C6 sugars, amino-functionalised C6 sugars, or combinations hereof.
19. The vaginal contraceptive composition according to any preceding item, wherein the monomer units are amino-functionalised C6 sugars.
20. The vaginal contraceptive composition according to any preceding item, wherein the monomer units are a combination of D-glucosamine and N-acetyl-D-glucosamine.
21. The vaginal contraceptive composition according to item 20, wherein at least 50% is D-glucosamine.
22. The vaginal contraceptive composition according to any of items 20-21, wherein 50% or less is N-acetyl-D-glucosamine.
23. The vaginal contraceptive composition according to any of items 20-22, wherein between 50% and 100% is D-glucosamine.
24. The vaginal contraceptive composition according to any of items 20-23, wherein between 0% and 50% is N-acetyl-D-glucosamine.
25. The vaginal contraceptive composition according to any of items 20-24, wherein at least 65% is D-glucosamine.
26. The vaginal contraceptive composition according to any of items 20-25, wherein 35% or less is N-acetyl-D-glucosamine.
27. The vaginal contraceptive composition according to any of items 20-26, wherein between 65% and 100% is D-glucosamine.
28. The vaginal contraceptive composition according to any of items 20-27, wherein between 0% and 35% is N-acetyl-D-glucosamine.
29. The vaginal contraceptive composition according to any of items 1-11, wherein the mucoadhesive polymer is a peptide molecule of a length of 469 to 4,661 amino acids, which are linked via amide bonds.
30. The vaginal contraceptive composition according to item 29, wherein the mucoadhesive polymer is a polypeptide of amino acids, wherein at least 50% of the amino acids are selected from the list consisting of arginine, lysine, histidine, ornithine, and β-alanine.
31. The vaginal contraceptive composition according to any of items 29-30, wherein the mucoadhesive polymer comprise poly-lysine, poly-orthinine, and/or poly-arginine.
32. The vaginal contraceptive composition according to item 31, wherein the mucoadhesive polymer comprise poly-lysine.
33. The vaginal contraceptive composition according to any of items 1-11, wherein the mucoadhesive polymer is a peptide molecule of a length of 469 to 4,661 amino acids wherein at least 50% of the amino acids carry a hydrophobic group, which amino acids are selected from the list consisting of: alanine, methionine, cysteine, phenylalanine, leucine, valine, and isoleucine, and wherein the remaining amino acids may be selected from the list consisting of: glycine, serine, threonine, asparagine, and glutamine.
34. The vaginal contraceptive composition according to any of items 1-11, wherein the mucoadhesive polymer comprises amino acids, wherein at least 50% of the amino acids are selected from the group consisting of arginine, lysine, histidine, ornithine, and β-alanine, or 50% of the amino acids carries a hydrophobic group, and are selected from the group consisting of alanine, methionine, cysteine, phenylalanine, leucine, valine, and isoleucine.
35. The vaginal contraceptive composition according to any of items 1-11, wherein the mucoadhesive polymer is a peptide molecule, which are linked via amide bonds, wherein at least 50% of the amino acids are selected from the list consisting of arginine, lysine, histidine, ornithine, and β-alanine.
36. The vaginal contraceptive composition according to item 35, wherein at least 60% of the amino acids are selected from the list consisting of arginine, lysine, histidine, ornithine, and β-alanine.
37. The vaginal contraceptive composition according to any of items 35-36, wherein at least 70% of the amino acids are selected from the list consisting of arginine, lysine, histidine, ornithine, and β-alanine.
38. The vaginal contraceptive composition according to any of items 1-11, wherein the mucoadhesive polymer is a peptide molecule, which are linked via amide bonds, wherein at least 50% of the amino acids are lysine.
39. The vaginal contraceptive composition according to item 38, wherein at least 60%, such as at least 70% of the amino acids are lysine.
40. The vaginal contraceptive composition according to any of items 1-11, wherein the mucoadhesive polymer comprises amino acids being L-lysine.
41. The vaginal contraceptive composition according to item 40, wherein the mucoadhesive polymer is poly-L-lysine (PLL).
42. The vaginal contraceptive composition according to any preceding item, wherein the physiological acceptable gelling agent is selected from hydroxyethyl cellulose (HEC), glycerol, hydroxypropylmethyl cellulose (HPMC), hydroxypropyl cellulose, guar gum, or combinations hereof.
43. The vaginal contraceptive composition according to any preceding item, wherein said mucoadhesive polymer consists of 616 to 2,054 monomer units linked to each other via ether bonds, ester bonds, amide bonds, or combinations hereof.
44. The vaginal contraceptive composition according to any preceding item, wherein said mucoadhesive polymer consists of 800 to 2,054 monomer units linked to each other via ether bonds, ester bonds, amide bonds, or combinations hereof.
45. The vaginal contraceptive composition according to any preceding item, wherein said mucoadhesive polymer consists of 800 to 1,800 monomer units linked to each other via ether bonds, ester bonds, amide bonds, or combinations hereof.
46. The vaginal contraceptive composition according to any preceding item, wherein said mucoadhesive polymer consists of 1,000 to 1,800 monomer units linked to each other via ether bonds, ester bonds, amide bonds, or combinations hereof.
47. The vaginal contraceptive composition according to any preceding item, wherein said mucoadhesive polymer consists of 1,200 to 1,600 monomer units linked to each other via ether bonds, ester bonds, amide bonds, or combinations hereof.
48. The vaginal contraceptive composition according to any preceding item, wherein the mucoadhesive polymer is in a concentration of between 0.05 wt. % and 10.0 wt. % of the total weight of the vaginal contraceptive composition.
49. The vaginal contraceptive composition according to any preceding item, wherein pH of the composition is between 2.0 and 7.0.
50. The vaginal contraceptive composition according to any preceding item, wherein pH of the composition is between 2.5 and 6.5.
51. The vaginal contraceptive composition according to any preceding item, wherein pH of the composition is between 3.0 and 6.0.
52. The vaginal contraceptive composition according to any preceding item, wherein the composition is a contraceptive composition.
53. Use of a vaginal contraceptive composition according to any of items 1-52 as a contraceptive agent.
54. A vaginal contraceptive composition for use in therapy, wherein the vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.
55. A vaginal contraceptive composition for use as a contraceptive or contraceptive agent, wherein the vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.
56. A vaginal contraceptive composition for use in birth control or birth control therapy, wherein the vaginal contraceptive composition comprises one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.
57. The vaginal contraceptive composition according to item 54 or 55 or 56, wherein the composition comprises one or more active ingredients, a physiological acceptable gelling agent, and a physiologically acceptable carrier.
58. The vaginal contraceptive composition according to any of items 54-57, wherein the physiological acceptable carrier is in a concentration of between 5.0 wt. % and 99.0 wt. % of the total weight of the vaginal contraceptive composition, such as between 5.0 wt. % and 90.0 wt. %, such as between 5.0 wt. % and 80.0 wt. %, such as between 10.0 wt. % and 75.0 wt. %, such as between 10.0 wt. % and 70.0 wt. %. such as between 10.0 wt. % and 60.0 wt. %, such as between 10.0 wt. % and 50.0 wt. %, such as at least 5.0 wt. %, or such as at least 10.0 wt. % of the total weight of the vaginal contraceptive composition.
59. The vaginal contraceptive composition according to any of items 54-58, wherein the physiological acceptable gelling agent is in a concentration of between 0.05 wt. % and 50.0 wt. % of the total weight of the vaginal contraceptive composition, such as between 0.10 wt. % and 50.0 wt. %, such as between 0.10 wt. % and 40.0 wt. %, such as between 0.10 wt. % and 30.0 wt. %. such as between 0.10 wt. % and 20.0 wt. %, such as between 0.05 wt. % and 10.0 wt. %, such as between 0.10 wt. % and 10.0 wt. %, or such as between 0.50 wt. % and 10.0 wt. % of the total weight of the vaginal contraceptive composition
60. The vaginal contraceptive composition according to any of items 54-59, wherein the vaginal contraceptive composition has an osmolality of at least 50 mOsm/kg, such as at least 75 mOsm/kg, or such as at least 100 mOsm/kg.
61. The vaginal contraceptive composition according to any of items 54-60, wherein the vaginal contraceptive composition is not a foam.
62. The vaginal contraceptive composition according to any of items 54-61, wherein the mucoadhesive polymer has a molecular weight between 100,000 Da and 350,000 Da, such as between 101,000 Da and 350,000 Da.
63. The vaginal contraceptive composition according to any of items 54-62, wherein the mucoadhesive polymer has a molecular weight between 110,000 Da and 350,000 Da, such as between 110,000 Da and 325,000 Da.
64. The vaginal contraceptive composition according to any of items 54-63, wherein the mucoadhesive polymer has a molecular weight between 120,000 Da and 350,000 Da, such as between 120,000 Da and 325,000 Da.
65. The vaginal contraceptive composition according to any of items 54-64, wherein the mucoadhesive polymer has a molecular weight between 125,000 Da and 350,000 Da, such as between 125,000 Da and 325,000 Da.
66. The vaginal contraceptive composition according to any of items 54-65, wherein the mucoadhesive polymer has a molecular weight between 101,000 Da and 149,000 Da or between 151,000 and 350,000 Da, such as between 101,000 Da and 149,000 Da or between 151,000 and 325,000 Da, such as between 110,000 Da and 149,000 Da or between 151,000 and 325,000 Da, such as between 120,000 Da and 149,000 Da or between 151,000 and 325,000 Da, or such as between 125,000 Da and 149,000 Da or between 151,000 and 325,000 Da.
67. The vaginal contraceptive composition according to any of items 54-66, wherein said mucoadhesive polymer consists of a plurality of monomer units linked to each other via ether bonds.
68. The vaginal contraceptive composition according to any of items 54-67, wherein at least 55% of the monomer units comprise at least one amino group.
69. The vaginal contraceptive composition according to any of items 54-68, wherein at least 60% of the monomer units comprise at least one amino group.
70. The vaginal contraceptive composition according to any of items 54-69, wherein at least 65% of the monomer units comprise at least one amino group.
71. The vaginal contraceptive composition according to any of items 54-70, wherein at least 70% of the monomer units comprise at least one amino group.
72. The vaginal contraceptive composition according to any of items 54-71, wherein the at least one amino group is a primary amine.
73. The vaginal contraceptive composition according to any of items 54-72, wherein the monomer units are selected from C6 sugars, amino-functionalised C6 sugars, or combinations hereof.
74. The vaginal contraceptive composition according to any of items 54-73, wherein the monomer units are amino-functionalised C6 sugars.
75. The vaginal contraceptive composition according to any of items 54-74, wherein the monomer units are a combination of D-glucosamine and N-acetyl-D-glucosamine.
76. The vaginal contraceptive composition according to item 75, wherein at least 50% is D-glucosamine.
77. The vaginal contraceptive composition according to any of items 75-76, wherein 50% or less is N-acetyl-D-glucosamine.
78. The vaginal contraceptive composition according to any of items 75-77, wherein between 50% and 100% is D-glucosamine.
79. The vaginal contraceptive composition according to any of items 75-78, wherein between 0% and 50% is N-acetyl-D-glucosamine.
80. The vaginal contraceptive composition according to any of items 75-79, wherein at least 65% is D-glucosamine.
81. The vaginal contraceptive composition according to any of items 75-80, wherein 35% or less is N-acetyl-D-glucosamine.
82. The vaginal contraceptive composition according to any of items 75-81, wherein between 65% and 100% is D-glucosamine.
83. The vaginal contraceptive composition according to any of items 75-82, wherein between 0% and 35% is N-acetyl-D-glucosamine.
84. The vaginal contraceptive composition according to any of items 54-66, wherein the mucoadhesive polymer is a peptide molecule of a length of 469 to 4,661 amino acids, which are linked via amide bonds.
85. The vaginal contraceptive composition according to item 84, wherein the mucoadhesive polymer is a polypeptide of amino acids, wherein at least 50% of the amino acids are selected from the list consisting of arginine, lysine, histidine, ornithine, and β-alanine.
86. The vaginal contraceptive composition according to any of items 84-85, wherein the mucoadhesive polymer comprise poly-lysine, poly-orthinine, and/or poly-arginine.
87. The vaginal contraceptive composition according to item 86, wherein the mucoadhesive polymer comprise poly-lysine.
88. The vaginal contraceptive composition according to any of items 54-66, wherein the mucoadhesive polymer is a peptide molecule of a length of 469 to 4,661 amino acids wherein at least 50% of the amino acids carry a hydrophobic group, which amino acids are selected from the list consisting of: alanine, methionine, cysteine, phenylalanine, leucine, valine, and isoleucine, and wherein the remaining amino acids may be selected from the list consisting of: glycine, serine, threonine, asparagine, and glutamine.
89. The vaginal contraceptive composition according to any of items 54-66, wherein the mucoadhesive polymer comprises amino acids, wherein at least 50% of the amino acids are selected from the group consisting of arginine, lysine, histidine, ornithine, and β-alanine, or 50% of the amino acids carries a hydrophobic group, and are selected from the group consisting of alanine, methionine, cysteine, phenylalanine, leucine, valine, and isoleucine.
90. The vaginal contraceptive composition according to any of items 54-66, wherein the mucoadhesive polymer is a peptide molecule, which are linked via amide bonds, wherein at least 50% of the amino acids are selected from the list consisting of arginine, lysine, histidine, ornithine, and β-alanine.
91. The vaginal contraceptive composition according to item 90, wherein at least 60% of the amino acids are selected from the list consisting of arginine, lysine, histidine, ornithine, and β-alanine.
92. The vaginal contraceptive composition according to any of items 90-91, wherein at least 70% of the amino acids are selected from the list consisting of arginine, lysine, histidine, ornithine, and β-alanine.
93. The vaginal contraceptive composition according to any of items 54-66, wherein the mucoadhesive polymer is a peptide molecule, which are linked via amide bonds, wherein at least 50% of the amino acids are lysine.
94. The vaginal contraceptive composition according to item 93, wherein at least 60%, such as at least 70% of the amino acids are lysine.
95. The vaginal contraceptive composition according to any of items 54-66, wherein the mucoadhesive polymer comprises amino acids being L-lysine.
96. The vaginal contraceptive composition according to item 95, wherein the mucoadhesive polymer is poly-L-lysine (PLL).
97. The vaginal contraceptive composition according to any of items 54-96, wherein the physiological acceptable gelling agent is selected from hydroxyethyl cellulose (HEC), glycerol, hydroxypropylmethyl cellulose (HPMC), hydroxypropyl cellulose, guar gum, or combinations hereof.
98. The vaginal contraceptive composition according to any of items 54-97, wherein said mucoadhesive polymer consists of 616 to 2,054 monomer units linked to each other via ether bonds, ester bonds, amide bonds, or combinations hereof.
99. The vaginal contraceptive composition according to any of items 54-98, wherein said mucoadhesive polymer consists of 800 to 2,054 monomer units linked to each other via ether bonds, ester bonds, amide bonds, or combinations hereof.
100. The vaginal contraceptive composition according to any of items 54-99, wherein said mucoadhesive polymer consists of 800 to 1,800 monomer units linked to each other via ether bonds, ester bonds, amide bonds, or combinations hereof.
101. The vaginal contraceptive composition according to any of items 54-100, wherein said mucoadhesive polymer consists of 1,000 to 1,800 monomer units linked to each other via ether bonds, ester bonds, amide bonds, or combinations hereof.
102. The vaginal contraceptive composition according to any of items 54-101, wherein said mucoadhesive polymer consists of 1,200 to 1,600 monomer units linked to each other via ether bonds, ester bonds, amide bonds, or combinations hereof.
103. The vaginal contraceptive composition according to any of items 54-102, wherein the mucoadhesive polymer is in a concentration of between 0.05 wt. % and 10.0 wt. % of the total weight of the vaginal contraceptive composition.
104. The vaginal contraceptive composition according to any of items 54-103, wherein pH of the composition is between 2.0 and 7.0.
105. The vaginal contraceptive composition according to any of items 54-104, wherein pH of the composition is between 2.5 and 6.5.
106. The vaginal contraceptive composition according to any of items 54-105, wherein pH of the composition is between 3.0 and 6.0.

1. A vaginal contraceptive composition comprising one or more active ingredients and at least one formulation compound selected from a physiological acceptable gelling agent or a physiological acceptable carrier, wherein at least one of the one or more active ingredients is a mucoadhesive polymer, wherein said mucoadhesive polymer has a molecular weight between 90,000 Da and 350,000 Da, wherein said mucoadhesive polymer consists of a plurality of monomer units linked to each other via ether bonds, ester bonds, amide bonds, or combinations hereof, wherein said monomer units are selected from C6 sugars, amino-functionalised C6 sugars, amino acids, or combinations hereof, and wherein at least 50% of the monomer units comprise at least one amino group.

2. The vaginal contraceptive composition according to claim 1, wherein the composition comprises one or more active ingredients, a physiological acceptable gelling agent, and a physiologically acceptable carrier.

3. The vaginal contraceptive composition according to claim 1, wherein the physiological acceptable carrier is in a concentration of between 5.0 wt. % and 99.0 wt. % of the total weight of the vaginal contraceptive composition, such as between 5.0 wt. % and 90.0 wt. %, such as between 5.0 wt. % and 80.0 wt. %, such as between 10.0 wt. % and 75.0 wt. %, such as between 10.0 wt. % and 70.0 wt. %. such as between 10.0 wt. % and 60.0 wt. %, such as between 10.0 wt. % and 50.0 wt. %, such as at least 5.0 wt. %, or such as at least 10.0 wt. % of the total weight of the vaginal contraceptive composition.

4. The vaginal contraceptive composition according to claim 1, wherein the physiological acceptable gelling agent is in a concentration of between 0.05 wt. % and 50.0 wt. % of the total weight of the vaginal contraceptive composition, such as between 0.10 wt. % and 50.0 wt. %, such as between 0.10 wt. % and 40.0 wt. %, such as between 0.10 wt. % and 30.0 wt. %. such as between 0.10 wt. % and 20.0 wt. %, such as between 0.05 wt. % and 10.0 wt. %, such as between 0.10 wt. % and 10.0 wt. %, or such as between 0.50 wt. % and 10.0 wt. % of the total weight of the vaginal contraceptive composition.

5. The vaginal contraceptive composition according to claim 1, wherein the vaginal contraceptive composition has an osmolality of at least 50 mOsm/kg, such as at least 75 mOsm/kg, or such as at least 100 mOsm/kg.

6. The vaginal contraceptive composition according to claim 1, wherein the vaginal contraceptive composition is not a foam.

7. The vaginal contraceptive composition according to claim 1, wherein the composition is a contraceptive composition.

8. The vaginal contraceptive composition according to claim 1, wherein the composition comprising one or more active ingredients, a physiological acceptable gelling agent, and a physiologically acceptable buffer solution.

9. The vaginal contraceptive composition according to claim 8, wherein the buffer solution is in a concentration from 1 mM to 350 mM, such as a concentration from 1 mM to 325 mM, such as a concentration from 1 mM to 300 mM, such as a concentration from 2 mM to 300 mM, such as a concentration from 3 mM to 300 mM, such as a concentration from 4 mM to 300 mM, such as a concentration from 5 mM to 300 mM, such as a concentration from 10 mM to 300 mM, such as a concentration from 15 mM to 300 mM, such as a concentration from 20 mM to 300 mM, such as a concentration from 20 mM to 250 mM, or such as a concentration from 20 mM to 200 mM.

10. The vaginal contraceptive composition according to claim 1, wherein, the composition comprising one or more active ingredients, a physiological acceptable gelling agent, and physiologically acceptable preservative.

11. The vaginal contraceptive composition according to claim 1, wherein the mucoadhesive polymer has a molecular weight between 101,000 Da and 350,000 Da; wherein the mucoadhesive polymer does not have a molecular weight of 150,000 Da, such as between 149,000 Da and 151,000 Da; and wherein the mucoadhesive polymer does not have a molecular weight between 251,000 Da and 252,000 Da, such as between 250,000 Da and 253,000 Da.

12. A method comprising: administering a vaginal contraceptive composition according to claim 1 to a mucous membrane of a subject as a contraceptive agent.

13-15. (canceled)

16. The method of claim 12, wherein the composition comprises one or more active ingredients, a physiological acceptable gelling agent, and a physiologically acceptable carrier.

17. The method of claim 12, wherein the physiological acceptable carrier is in a concentration of between 5.0 wt. % and 99.0 wt. % of the total weight of the vaginal contraceptive composition, such as between 5.0 wt. % and 90.0 wt. %, such as between 5.0 wt. % and 80.0 wt. %, such as between 10.0 wt. % and 75.0 wt. %, such as between 10.0 wt. % and 70.0 wt. %. such as between 10.0 wt. % and 60.0 wt. %, such as between 10.0 wt. % and 50.0 wt. %, such as at least 5.0 wt. %, or such as at least 10.0 wt. % of the total weight of the vaginal contraceptive composition.

18. The method of claim 12, wherein the physiological acceptable gelling agent is in a concentration of between 0.05 wt. % and 50.0 wt. % of the total weight of the vaginal contraceptive composition, such as between 0.10 wt. % and 50.0 wt. %, such as between 0.10 wt. % and 40.0 wt. %, such as between 0.10 wt. % and 30.0 wt. %. such as between 0.10 wt. % and 20.0 wt. %, such as between 0.05 wt. % and 10.0 wt. %, such as between 0.10 wt. % and 10.0 wt. %, or such as between 0.50 wt. % and 10.0 wt. % of the total weight of the vaginal contraceptive composition

19. The method of claim 12, wherein the vaginal contraceptive composition has an osmolality of at least 50 mOsm/kg, such as at least 75 mOsm/kg, or such as at least 100 mOsm/kg.

20. The method of claim 12, wherein the vaginal contraceptive composition is not a foam.

21. The method of claim 12, wherein the composition comprising one or more active ingredients, a physiological acceptable gelling agent, and a physiologically acceptable buffer solution.

22. The method of claim 12, wherein, the composition comprising one or more active ingredients, a physiological acceptable gelling agent, and physiologically acceptable preservative.

23. The method of claim 12, wherein the mucoadhesive polymer has a molecular weight between 101,000 Da and 350,000 Da; wherein the mucoadhesive polymer does not have a molecular weight of 150,000 Da, such as between 149,000 Da and 151,000 Da; and wherein the mucoadhesive polymer does not have a molecular weight between 251,000 Da and 252,000 Da, such as between 250,000 Da and 253,000 Da.
